(19) United States
(12) Patent Application Publication
(10) Pub. No.: US 2006/0030566 A1
(43) Pub. Date: Feb. 9, 2006

(54) Novel quinoxalinone norepinephrine reuptake inhibitors for the treatment of central nervous system disorders

(75) Inventors: Robert M. Schelkun, Milan, MI (US); Po-wai Yuen, Ann Arbor, MI (US)

Correspondence Address: WARNER-LAMBERT COMPANY, 2800 PLYMOUTH RD, ANN ARBOR, MI, 48105, US

(73) Assignee: Warner-Lambert Company, LLC

(21) Appl. No.: 11/199,056

(22) Filed: Aug. 8, 2005

Related U.S. Application Data
(60) Provisional application No. 60/599,997, filed on Aug. 9, 2004.

(51) Int. Cl. A61K 31/498; C07D 403/04

(52) U.S. Cl. 514/249; 544/354

(57) ABSTRACT

This invention relates to compounds of the formula I 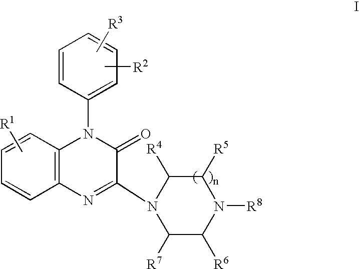
wherein R¹-R⁸ and n are defined as in the specification, pharmaceutical compositions containing them and their use in the treatment of central nervous system disorders.

This application is a U.S. utility application, which claims the benefit of priority to U.S. Provisional Application No. 60/599,997 filed Aug. 9, 2004.

This invention relates to a method of preventing or treating central nervous system disorders or conditions and in particular a method of treating or preventing attention deficit hyperactivity disorder (“ADHD”) by administering a compound that inhibits the reuptake of norepinephrine. Such compounds are also referred to in the literature as selective norepinephrine reuptake inhibitors (NRIs).

### BACKGROUND OF THE INVENTION

Attention deficit hyperactivity disorder (ADHD) has an estimated incidence in school age children of 3-5%, and is characterized by the core symptoms of hyperactivity, impulsivity, and/or inattention. The attentional symptoms of ADHD can be successfully treated with psychomotor stimulants such as methylphenidate (Ritalin). Clonidine, an α₂-adrenoceptor agonist, treats the aggressive and oppositional 2 symptoms. There is a potential for significant side effects with both methylphenidate and clonidine, making it important to identify other drugs that have similar or better efficacy with reduced side effects and abuse liability.

ADHD is one of the most common childhood psychiatric disorders and appears to be a common, often underrecognized, psychiatric disease in adults as well (T. Spencer, et al., J Clin Psychiatry, 1998, 59 (Suppl. 7), 759-768). This disorder, which begins in childhood, may be followed by a lifelong expression of symptoms (e.g., inattention and/or impulsivity) (JB. Schweitzer, et al., Med Clin North Am, May 2001, 85:3, 757-777). ADHD may change its manifestations as it develops from preschool through adult life (DP. Cantwell, J Am Acad Child Adolesc Psychiatry, August 1996, 35(8), 978-987; J. Elia, et al. N Eng J Med, March 1999, 340(10), 780-788; EE. Nolan, et al., J Am Acad Child Adolesc Psychaitry, February 2001, 40(2), 241-249).

The diagnosis of ADHD is based on clinical evaluation (M. Dulcan, et al. J Am Acad Child Adolesc Psychaitry, October 1997, 36(10 Suppl), 85S-121S; National Institutes of Health, 1998). “The essential feature of ADHD is a persistent pattern of inattention and/or hyperactivity-impulsivity that is more frequent and severe than is typically observed in individuals at a comparative level of development” (Diagnostic and Statistical Manual of Mental Disorders (DSM-IV), American Psychiatric Association, Washington, D.C., 1994). In order to be diagnosed with ADHD, patients must demonstrate symptoms of ADHD that cause impairment before the age of seven years, and symptoms must have been ongoing for longer than six months in at least two settings (e.g., school [or work] and home). (See DSM-IV).

Several NRI compounds are known. Atomoxetine, an NRI, is now commercially available (Strattera®, Eli Lilly) and is beginning to be used extensively for the clinical treatment of ADHD in both children and adults. Atomoxetine represents a non-stimulant treatment for ADHD. The number of treated ADHD patients is expected to increase as a result of the introduction of atomoxetine and enhanced educational initiatives. Accordingly, there is an ongoing need for ADHD treatments that provide more efficacy than those treatments currently available.

### SUMMARY OF THE INVENTION

The present invention relates to compounds of the formula I 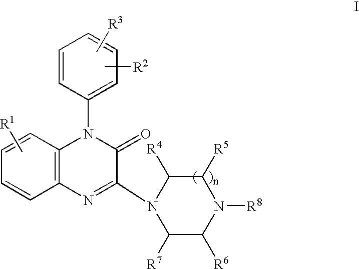
and pharmaceutically acceptable salts or derivatives thereof, wherein:
R¹ is H, (C₁-C₆)alkyl, or halogen;
R² and R³ are independently selected from is H, (C₁-C₆)alkyl, or halogen;
n is 1 or 2;
R⁴-R⁸ are independently selected from H and (C₁-C₈)alkyl.

A further embodiment of the invention relates to compounds wherein R2 is a halogen.

Yet a further embodiment of the invention relates to compounds wherein R² and R³ are fluoro.

Yet a further embodiment of the invention relates to compounds wherein R¹ is fluoro, n is 1 and R⁸=H.

Yet a further embodiment of the invention relates to compounds wherein R¹ is fluoro, R² and R³ are fluoro, n is 1, and R⁸=H.

Preferred compounds of the invention include the following compounds and their pharmaceutically acceptable salts:
1-Phenyl-3-piperazin-1-yl-1H-quinoxalin-2-one hydrochloride;
1-(2-Fluoro-phenyl)-3-piperazin-1-yl-1H-quinoxalin-2-one hydrochloride;
1-(2-Chloro-phenyl)-3-piperazin-1-yl-1H-quinoxalin-2-one;
3-Piperazin-1-yl-1-o-tolyl-1H-quinoxalin-2-one;
1-(3-Fluoro-phenyl)-3-piperazin-1-yl-1H-quinoxalin-2-one;
3-Piperazin-1-yl-1-p-tolyl-1H-quinoxalin-2-one maleate;
7-Fluoro-1-phenyl-3-piperazin-1-yl-1H-quinoxalin-2-one;
7-Methyl-1-phenyl-3-piperazin-1-yl-1H-quinoxalin-2-one;
6-Fluoro-1-phenyl-3-piperazin-1-yl-1H-quinoxalin-2-one;
1-(2,6-Difluoro-phenyl)-6-fluoro-3-piperazin-1 -yl-1H-quinoxalin-2-one;
1-(2-Chloro-phenyl)-3-[1,4]diazepan-1-yl-1H-quinoxalin-2-one maleate;
3-[1,4]Diazepan-1-yl-1-(3-fluoro-phenyl)-1H-quinoxalin-2-one;
3-[1,4]Diazepan-1-yl-1-p-tolyl-1H-quinoxalin-2-one maleate;
3-[1,4]Diazepan-1-yl-6-fluoro-1-phenyl-1H-quinoxalin-2-one; and
3-[1,4]Diazepan-1-yl-1-isopropyl-1H-quinoxalin-2-one maleate.

The present invention further relates to a method of treatment of attention deficit hyperactivity disorder, urinary disorders, pain, anxiety, depression, premature ejaculation, or fibromyalgia, which comprises administering a therapeutically effective amount of a compound as defined in any of formulae I.

This invention also relates to a method of treating a disorder or condition selected from the group consisting of norepinephrine dysfunction, single episodic or recurrent major depressive disorders, dysthymic disorders, depressive neurosis and neurotic depression, melancholic depression including anorexia, weight loss, insomnia, early morning waking or psychomotor retardation; atypical depression (or reactive depression) including increased appetite, hypersomnia, psychomotor agitation or irritability, seasonal affective disorder and pediatric depression; bipolar disorders or manic depression, for example, bipolar I disorder, bipolar II disorder and cyclothymic disorder; conduct disorder; attention deficit hyperactivity disorder (ADHD); disruptive behavior disorder; behavioral disturbances associated with mental retardation, autistic disorder, and conduct disorder; anxiety disorders such as panic disorder with or without agoraphobia, agoraphobia without history of panic disorder, specific phobias, for example, specific animal phobias, social anxiety, social phobia (including social anxiety disorder), obsessive-compulsive disorder and related spectrum disorders, stress disorders including post-traumatic stress disorder, acute stress disorder and chronic stress disorder, and generalized anxiety disorders; borderline personality disorder; schizophrenia and other psychotic disorders, for example, schizophreniform disorders, schizoaffective disorders, delusional disorders, brief psychotic disorders, shared psychotic disorders, psychotic disorders with delusions or hallucinations, psychotic episodes of anxiety, anxiety associated with psychosis, psychotic mood disorders such as severe major depressive disorder; mood disorders associated with psychotic disorders such as acute mania and depression associated with bipolar disorder; mood disorders associated with schizophrenia; delirium, dementia, and amnestic and other cognitive or neurodegenerative disorders, such as Parkinson's disease (PD), Huntington's disease (HD), Alzheimer's disease, senile dementia, dementia of the Alzheimer's type, memory disorders, loss of executive function, vascular dementia, and other dementias, for example, due to HIV disease, head trauma, Parkinson's disease, Huntington's disease, Pick's disease, Creutzfeldt-Jakob disease, or due to multiple etiologies; movement disorders such as akinesias, dyskinesias, including familial paroxysmal dyskinesias, spasticities, Tourette's syndrome, Scott syndrome, PALSYS and akinetic-rigid syndrome; extra-pyramidal movement disorders such as medication-induced movement disorders, for example, neuroleptic-induced Parkinsonism, neuroleptic malignant syndrome, neuroleptic-induced acute dystonia, neuroleptic-induced acute akathisia, neuroleptic-induced tardive dyskinesia and medication-induced postural tremour; addictive disorders and withdrawal syndrome, chemical dependencies and addictions (e.g., dependencies on, or addictions to, alcohol, heroin, cocaine, benzodiazepines, sychoactive substances, nicotine, or phenobarbitol) and behavioral addictions such as an addiction to gambling; ocular disorders such as glaucoma and ischemic retinopathy addictive disorders (including those due to alcohol, nicotine, and other psychoactive substances) and withdrawal syndrome, adjustment disorders (including depressed mood, anxiety, mixed anxiety and depressed mood, disturbance of conduct, and mixed disturbance of conduct and mood); age-associated learning and mental disorders (including Alzheimer's disease); anorexia nervosa; apathy; attention-deficit (or other cognitive) disorders due to general medical conditions including attention-deficit disorder (ADD) and attention-deficit hyperactivity disorder (ADHD) and it's recognized sub-types; bulimia nervosa; chronic fatigue syndrome; pain; chronic pain; cyclothymic disorder; depression (including adolescent depression and minor depression); fibromyalgia and other somatoform disorders (including somatization disorder; conversion disorder; pain disorder; hypochondriasis; body dysmorphic disorder; undifferentiated somatoform disorder; and somatoform NOS); incontinence (i.e.; stress incontinence; genuine stress incontinence; and mixed incontinence); urinary disorders; premature ejaculation; inhalation disorders; intoxication disorders (alcohol addiction); mania; migraine headaches; obesity (i.e.; reducing the weight of obese or overweight patients); restless legs syndrome; oppositional defiant disorder; peripheral neuropathy; diabetic neuropathy; post-herpetic neuralgic; premenstrual dysphoric disorder (i.e.; premenstrual syndrome and late luteal phase dysphoric disorder); hot flashes; sleep disorders (such as narcolepsy, insomnia and enuresis); specific developmental disorders; selective serotonin reuptake inhibition (SSRI) “poop out” syndrome (i.e.; wherein a patient who fails to maintain a satisfactory response to SSRI therapy after an initial period of satisfactory response); and TIC disorders (e.g.; Tourette's Disease) in a mammal, including a human, comprising administering to a mammal in need of such treatment an amount of a compound of the formula I or a pharmaceutically acceptable salt thereof, that is effective in treating such disorder or condition.

This invention also relates to a pharmaceutical composition comprising a therapeutically effective amount of a compound of the formula I, or a pharmaceutically acceptable salt thereof, and a pharmaceutically acceptable carrier.

This invention also relates to a pharmaceutical composition for treating a disorder or condition selected from norepinephrine dysfunction, single episodic or recurrent major depressive disorders, dysthymic disorders, depressive neurosis and neurotic depression, melancholic depression including anorexia, weight loss, insomnia, early morning waking or psychomotor retardation; atypical depression (or reactive depression) including increased appetite, hypersomnia, psychomotor agitation or irritability, seasonal affective disorder and pediatric depression; bipolar disorders or manic depression, for example, bipolar I disorder, bipolar II disorder and cyclothymic disorder; conduct disorder; attention deficit hyperactivity disorder (ADHD); disruptive behavior disorder; behavioral disturbances associated with mental retardation, autistic disorder, and conduct disorder; anxiety disorders such as panic disorder with or without agoraphobia, agoraphobia without history of panic disorder, specific phobias, for example, specific animal phobias, social anxiety, social phobia (including social anxiety disorder), obsessive-compulsive disorder and related spectrum disorders, stress disorders including post-traumatic stress disorder, acute stress disorder and chronic stress disorder, and generalized anxiety disorders; borderline personality disorder; schizophrenia and other psychotic disorders, for example, schizophreniform disorders, schizoaffective disorders, delusional disorders, brief psychotic disorders, shared psychotic disorders, psychotic disorders with delusions or hallucinations, psychotic episodes of anxiety, anxiety associated with psychosis, psychotic mood disorders such as severe major depressive disorder; mood disorders associated with psychotic disorders such as acute mania and depression associated with bipolar disorder; mood disorders associated with schizophrenia; delirium, dementia, and amnestic and other cognitive or neurodegenerative disorders, such as Parkinson's disease (PD), Huntington's disease (HD), Alzheimer's disease, senile dementia, dementia of the Alzheimer's type, memory disorders, loss of executive function, vascular dementia, and other dementias, for example, due to HIV disease, head trauma, Parkinson's disease, Huntington's disease, Pick's disease, Creutzfeldt-Jakob disease, or due to multiple etiologies; movement disorders such as akinesias, dyskinesias, including familial paroxysmal dyskinesias, spasticities, Tourette's syndrome, Scott syndrome, PALSYS and akinetic-rigid syndrome; extra-pyramidal movement disorders such as medication-induced movement disorders, for example, neuroleptic-induced Parkinsonism, neuroleptic malignant syndrome, neuroleptic-induced acute dystonia, neuroleptic-induced acute akathisia, neuroleptic-induced tardive dyskinesia and medication-induced postural tremour; addictive disorders and withdrawal syndrome, chemical dependencies and addictions (e.g., dependencies on, or addictions to, alcohol, heroin, cocaine, benzodiazepines, sychoactive substances, nicotine, or phenobarbitol) and behavioral addictions such as an addiction to gambling; ocular disorders such as glaucoma and ischemic retinopathy addictive disorders (including those due to alcohol, nicotine, and other psychoactive substances) and withdrawal syndrome, adjustment disorders (including depressed mood, anxiety, mixed anxiety and depressed mood, disturbance of conduct, and mixed disturbance of conduct and mood); age-associated learning and mental disorders (including Alzheimer's disease); anorexia nervosa; apathy; attention-deficit (or other cognitive) disorders due to general medical conditions including attention-deficit disorder (ADD) and attention-deficit hyperactivity disorder (ADHD) and it's recognized sub-types; restless legs syndrome bulimia nervosa; chronic fatigue syndrome; pain; chronic pain; cyclothymic disorder; depression (including adolescent depression and minor depression); fibromyalgia and other somatoform disorders (including somatization disorder; conversion disorder; pain disorder; hypochondriasis; body dysmorphic disorder; undifferentiated somatoform disorder; and somatoform NOS); incontinence (i.e.; stress incontinence; genuine stress incontinence; and mixed incontinence); urinary disorders; premature ejaculation; inhalation disorders; intoxication disorders (alcohol addiction); mania; migraine headaches; obesity (i.e.; reducing the weight of obese or overweight patients); oppositional defiant disorder; peripheral neuropathy; diabetic neuropathy; post-herpetic neuralgic; premenstrual dysphoric disorder (i.e.; premenstrual syndrome and late luteal phase dysphoric disorder); hot flashes; sleep disorders (such as narcolepsy, insomnia and enuresis); specific developmental disorders; selective serotonin reuptake inhibition (SSRI) “poop out” syndrome (i.e.; wherein a patient who fails to maintain a satisfactory response to SSRI therapy after an initial period of satisfactory response); and TIC disorders (e.g.; Tourette's Disease) in a mammal in need of such treatment, including a human, comprising an amount of a compound of the formula I or a pharmaceutically acceptable salt thereof, that is effective in treating such disorder or condition, and a pharmaceutically acceptable carrier.

Another specific embodiment of this invention relates to the above method wherein the compound of formula I is administered to a human for the treatment of any two or more comorbid disorders or conditions selected from those disorders and conditions referred to in any of the above methods.

For the treatment of ADHD, depression, anxiety, schizophrenia or any of the other disorders and conditions referred to above in the descriptions of the methods and pharmaceutical compositions of this invention, the novel compounds of this invention can be used in conjunction with one or more additional active agents including antidepressants, anti-psychotics or anti-anxiety agents. Examples of classes of antidepressants that can be used in combination with the active compounds of this invention include norepinephrine reuptake inhibitors (NRIs), selective serotonin reuptake inhibitors (SRIs), NK-1 receptor antagonists, monoamine oxidase inhibitors (MAOIs), reversible inhibitors of monoamine oxidase (RIMAs), dual serotonin and norepinephrine reuptake inhibitors, corticotropin releasing factor (CRF) antagonists, α-adrenoreceptor antagonists, alpha-2-delta ligands (A2D), and atypical antidepressants.

Another type of agent that can be used in combination with the novel compounds of this invention are nicotinic receptor agonists or antagonists.

SRIs useful for the methods and pharmaceutical compositions of the present invention include, but are not limited to sertraline (Zoloft®), sertraline metabolite demethylsertraline, fluoxetine (Prozac®), norfluoxetine (fluoxetine desmethyl metabolite), fluvoxamine (Luvox®, paroxetine (Seroxat®, Paxil®) and its alternative formulation, Paxil-CR®, citalopram (Celexa®), citalopram metabolite desmethylcitalopram, escitalopram (Lexapro®), d,l-fenfluramine (Pondimin®), femoxetine, ifoxetine, cyanodothiepin, litoxetine, cericlamine, dapoxetine, nefazaodone (Serxone®), and trazodone (Desyrel®), or any prodrug thereof or any pharmaceutically acceptable salt of the SRI or the prodrug thereof.

NRIs useful for the methods and pharmaceutical compositions of the present invention include, but are not limited to, reboxetine (Edronax®) and all isomers of reboxetine, ie., (R/R,S/S,R/S,S/R), desipramine (Norpramin®), maprotiline (Ludiomil®), Iofepramine (Gamanil®), mirtazepine (Remeron®), oxaprotiline, fezolamine, atomoxetine (Strattera®) and buproprion (Wellbutrin®), buproprion metabolite hydroxybuproprion, nomifensine (Merital®), viloxazine (Vivalan®), or mianserin (Bolvidon®) or any prodrug thereof or any pharmaceutically acceptable salt of the NRI or the prodrug thereof.

Pharmaceutical agents, which inhibit the reuptake of both serotonin and norepinephrine include venlafaxine (Effexor®), venlafaxine metabolite O-desmethylvenlafaxine, clomipramine (Anafranil®), clomipramine metabolite desmethylclomipramine, duloxetine (Cymbalta®), milnacipran, and imipramine (Tofranil® or Janimine®).

Examples of preferred A2D ligands for use with the present invention are those compounds generally or specifically disclosed in U.S. Pat. No. 4,024,175, particularly gabapentin, EP641330, particularly pregabalin, U.S. Pat. No. 5,563,175, WO9733858, WO9733859, WO9931057, WO9931074, WO9729101, WO02085839, particularly [(1R,5R,6S)-6-(Aminomethyl)bicyclo[3.2.0]hept-6-yl] acetic acid, WO9931075, particularly 3-(1-Aminomethyl-cyclohexylmethyl)-4H-[1,2,4]oxadiazol-5-one and C-[1-(1H-Tetrazol-5-ylmethyl)-cycloheptyl]-methylamine, WO9921824, particularly (3S,4S)-(1-Aminomethyl-3,4-dimethyl-cyclopentyl)-acetic acid, WO0190052, WO0128978, particularly (1a,3a,5a)(3-amino-methyl-bicyclo[3.2.0]hept-3-yl)-acetic acid, EP0641330, WO9817627, WO0076958, particularly (3S,5R)-3-aminomethyl-5-methyl-octanoic acid, U.S. Ser. No. 10/401,060, particularly (3S,5R)-3-amino-5-methyl-heptanoic acid, U.S. Ser. No. 10/401,060, (3S,5R)-3-amino-5-methyl-nonanoic acid, and (3S,5R)-3-Amino-5-methyl-octanoic acid, EP1178034, EP1201240, WO9931074, WO03000642, WO0222568, WO0230871, WO0230881 WO02100392, WO02100347, WO0242414, WO0232736 and WO0228881, and pharmaceutically acceptable salts and solvates thereof.

Suitable CRF antagonists include those compounds described in International Patent Application Nos. WO 94/13643, WO 94/13644, WO 94/13661, WO 94/13676 and WO 94/13677. Suitable atypical anti-depressants include bupropion, lithium, nefazodone, trazodone and viloxazine.

Suitable NK-1 receptor antagonists include those referred to in World Patent Publication WO 01/77100.

Suitable classes of anti-anxiety agents that can be used in combination with the active compounds of this invention include benzodiazepines and serotonin IA (5-HT_{IA}) agonists or antagonists, especially 5-HT_{IA} partial agonists, and corticotropin releasing factor (CRF) antagonists. Suitable benzodiazepines include alprazolam, chlordiazepoxide, clonazepam, chlorazepate, diazepam, halazepam, lorazepam, oxazepam, and prazepam. Suitable 5-HT_{IA} receptor agonists or antagonists include buspirone, flesinoxan, gepirone and ipsapirone.

Suitable antipsychotic agents include both conventional and atypical antipsychotics.

Conventional antipsychotics are antagonists of dopamine (D₂) receptors. The atypical antipsychotics also have D₂ antagonistic properties but possess different binding kinetics to these receptors and activity at other receptors, particularly 5-HT_{2A}, 5-HT_{2C} and 5-HT_{2D} (Schmidt B et al, Soc. Neurosci. Abstr. 24:2177, 1998).

Examples of dopamine (D4) receptor ligands are described in U.S. Pat. No. 6,548,502, U.S. Pat. No. 5,852,031, U.S. Pat. No. 5,883,094, U.S. Pat. No. 5,889,010 and WO 98/08835.

Examples of nicotinic receptor agonists or antagonists include: varenicline, azaindole-ethylamine derivatives as described in U.S. Pat. No. 5,977,131, and analogs, derivatives, prodrugs, and pharmaceutically acceptable salts of the nicotinic receptor agonists or antagonists and the prodrugs.

A particularly preferred nicotinic receptor agonist is varenicline, 7, 8, 9, 10-tetrahydro-6,10-methano-6H-pyrazino[2,3-h][3]benzapine (2R, 3R)-2,3-dihydroxybutanedioate, or any pharmaceutical acceptable salt thereof, including any polymorph or any prodrug thereof, or any pharmaceutically acceptable salt of such prodrug. A preferred salt of varenicline is varenicline tartrate. Varenicline is a partial nicotine agonist with affinity for some nicotine receptor subtypes but not others. Synthesis of varenicline tartrate is disclosed in WO 99/35131, U.S. Pat. No. 6,410,550, Patent Application Nos. 1997070245, 2002072524, 2002072525, 2002111350, and 2002132824.

The class of atypical antipsychotics includes clozapine (Clozaril®), 8-chloro-11-(4-methyl-1-piperazinyl)-5H-dibenzo[b,e][1,4]diazepine (U.S. Pat. No. 3,539,573); risperidone (Risperdal®), 3-[2-[4-(6-fluoro-1,2-benzisoxazol-3-yl)piperidino]ethyl]-2-methyl-6,7,8,9-tetrahydro-4H-pyrido-[1,2-a]pyrimidin-4-one (U.S. Pat. No. 4,804,663); olanzapine (Zyprexa®), 2-methyl-4-(4-methyl-1-piperazinyl)-10H-thieno[2,3-b][1,5]benzodiazepine (U.S. Pat. No. 5,229,382); quetiapine (Seroquel®), 5-[2-(4-dibenzo[b,f][1,4]thiazepin-11-yl-1-piperazinyl)ethoxy]ethanol (U.S. Pat. No. 4,879,288); aripiprazole (Abilify®), 7-{4-[4-(2,3-dichlorophenyl)-1-piperazinyl]-butoxy}-3,4-dihydro carbostyril and 7-{4-[4-(2,3-dichlorophenyl)-1-piperazinyl]-butoxy}-3,4-dihydro-2(1H)-quinolinone (U.S. Pat. Nos. 4,734,416 and 5,006,528); sertindole, 1-[2-[4-[5-chloro-1-(4-fluorophenyl)-1H-indol-3-yl]-1-piperidinyl]ethyl]imidazolidin-2-one (U.S. Pat. No. 4,710,500); amisulpride (U.S. Pat. No. 4,410,822); ziprasidone (Geodon®) 5-[2-[4-(1,2-benzisothiazol-3-yl)piperazin-3-yl]ethyl]-6-chloroindolin-2-one hydrochloride hydrate (U.S. Pat. No. 4,831,031); and asenapinetrans-5-chloro-2-methyl-2,3,3a,12b-tetrahydro-1H- dibenz[2,3:6,7]oxepino[4,5-c]pyrrole (U.S. Pat. Nos. 4,145,434 and 5,763,476).

This invention also relates to a method of treating a disorder or condition selected from addictive disorders (including those due to alcohol, nicotine, and other psychoactive substances) and withdrawal syndrome, adjustment disorders (including depressed mood, anxiety, mixed anxiety and depressed mood, disturbance of conduct, and mixed disturbance of conduct and mood); age-associated learning and mental disorders (including Alzheimer's disease); anorexia nervosa; apathy; attention-deficit (or other cognitive) disorders due to general medical conditions including attention-deficit disorder (ADD) and attention-deficit hyperactivity disorder (ADHD) and it's recognized sub-types; restless legs syndrome; bulimia nervosa; chronic fatigue syndrome; pain; chronic pain; cyclothymic disorder; depression (including adolescent depression and minor depression); fibromyalgia and other somatoform disorders (including somatization disorder; conversion disorder; pain disorder; hypochondriasis; body dysmorphic disorder; undifferentiated somatoform disorder; and somatoform NOS); incontinence (i.e.; stress incontinence; genuine stress incontinence; and mixed incontinence); urinary disorders; premature ejaculation; inhalation disorders; intoxication disorders (alcohol addiction); mania; migraine headaches; obesity (i.e.; reducing the weight of obese or overweight patients); oppositional defiant disorder; peripheral neuropathy; diabetic neuropathy; post-herpetic neuralgic; premenstrual dysphoric disorder (i.e.; premenstrual syndrome and late luteal phase dysphoric disorder); hot flashes; sleep disorders (such as narcolepsy, insomnia and enuresis); specific developmental disorders; selective serotonin reuptake inhibition (SSRI) “poop out” syndrome (i.e.; wherein a patient who fails to maintain a satisfactory response to SSRI therapy after an initial period of satisfactory response); and TIC disorders (e.g.; Tourette's Disease) in a mammal in need of such treatment, including a human, comprising administering to said mammal:
(a) a compound of the formula I or a pharmaceutically acceptable salt thereof; and
(b) another pharmaceutically active compound that is an anti-depressant, anti-psychotic or anti-anxiety agent, or a pharmaceutically acceptable salt thereof;
   wherein the active compounds “a” and “b” are present in amounts that render the combination effective in treating such disorder or condition.

Another more specific embodiment of this invention relates to the above method wherein the compounds of formula I and the additional pharmaceutical agent includes an antidepressant, anti-anxiety agent, or anti- psychotic are administered to a human for the treatment of any two or more comorbid disorders or conditions selected from those disorders and conditions referred to in any of the above methods.

This invention also relates to a pharmaceutical composition for treating a disorder or condition selected from addictive disorders (including those due to alcohol, nicotine, and other psychoactive substances) and withdrawal syndrome, adjustment disorders (including depressed mood, anxiety, mixed anxiety and depressed mood, disturbance of conduct, and mixed disturbance of conduct and mood); age-associated learning and mental disorders (including Alzheimer's disease); anorexia nervosa; apathy; attention-deficit (or other cognitive) disorders due to general medical conditions including attention-deficit disorder (ADD) and attention-deficit hyperactivity disorder (ADHD) and it's recognized sub-types; bulimia nervosa; chronic fatigue syndrome; pain; chronic pain; cyclothymic disorder; depression (including adolescent depression and minor depression); fibromyalgia and other somatoform disorders (including somatization disorder; conversion disorder; pain disorder; hypochondriasis; body dysmorphic disorder; undifferentiated somatoform disorder; and somatoform NOS); incontinence (i.e.; stress incontinence; genuine stress incontinence; and mixed incontinence); urinary disorders; premature ejaculation; inhalation disorders; intoxication disorders (alcohol addiction); mania; migraine headaches; obesity (i.e.; reducing the weight of obese or overweight patients); restless legs syndrome; oppositional defiant disorder; peripheral neuropathy; diabetic neuropathy; post-herpetic neuralgic; premenstrual dysphoric disorder (i.e.; premenstrual syndrome and late luteal phase dysphoric disorder); hot flashes; sleep disorders (such as narcolepsy, insomnia and enuresis); specific developmental disorders; selective serotonin reuptake inhibition (SSRI) “poop out” syndrome (i.e.; wherein a patient who fails to maintain a satisfactory response to SSRI therapy after an initial period of satisfactory response); and TIC disorders (e.g.; Tourette's Disease) in a mammal in need of such treatment, including a human, comprising:
(a) a compound of the formula I or a pharmaceutically acceptable salt thereof;
(b) another pharmaceutically active compound including an antidepressant or anti-anxiety agent, or a pharmaceutically acceptable salt thereof; and
(c) a pharmaceutically acceptable carrier;

wherein the active compounds “a” and “b” are present in amounts that render the composition effective in treating such disorder or condition.

This invention also relates to a pharmaceutical composition comprising a therapeutically effective amount of a compound of the formula I or a pharmaceutically acceptable salt thereof, and a pharmaceutically acceptable carrier.

Compounds of formula I may contain chiral centers and therefore may exist in different enantiomeric and diastereomeric forms. This invention relates to all optical isomers and all stereoisomers of compounds of the formula I, both as racemic mixtures and as individual enantiomers and diastereoisomers of such compounds, and mixtures thereof, and to all pharmaceutical compositions and methods of treatment defined above that contain or employ them, respectively. Individual isomers can be obtained by known methods, such as classical resolution, stereo-selective reaction, or chromatographic separation in the preparation of the final product or its intermediate. Individual enantiomers of the compounds of formula I may have advantages, as compared with the racemic mixtures of these compounds, in the treatment of various disorders or conditions.

In so far as the compounds of formula I of this invention are basic compounds, they are all capable of forming a wide variety of different salts with various inorganic and organic acids. Although such salts must be pharmaceutically acceptable for administration to animals, it is often desirable in practice to initially isolate the base compound from the reaction mixture as a pharmaceutically unacceptable salt and then simply convert to the free base compound by treatment with an alkaline reagent and thereafter convert the free base to a pharmaceutically acceptable acid addition salt. The acid addition salts of the base compounds of this invention are readily prepared by treating the base compound with a substantially equivalent amount of the chosen mineral or organic acid in an aqueous solvent or in a suitable organic solvent, such as methanol or ethanol. Upon careful evaporation of the solvent, the desired solid salt is readily obtained. The acids which are used to prepare the pharmaceutically acceptable acid addition salts of the aforementioned base compounds of this invention are those which form non-toxic acid addition salts, i.e., salts containing pharmaceutically acceptable anions, such as the hydrochloride, hydrobromide, hydroiodide, nitrate, sulfate or bisulfate, phosphate or acid phosphate, acetate, lactate, citrate or acid citrate, tartrate or bi-tartrate, succinate, maleate, fumarate, gluconate, saccharate, benzoate, methanesulfonate, ethanesulfonate, benzenesulfonate, p-toluenesulfonate and pamoate (i.e., 1,1′-methylene-bis-(2-hydroxy-3-naphthoate)) salts.

The present invention also includes isotopically labelled compounds, which are identical to those recited in formula 1, but for the fact that one or more atoms are replaced by an atom having an atomic mass or mass number different from the atomic mass or mass number usually found in nature. Examples of isotopes that can be incorporated into compounds of the present invention include isotopes of hydrogen, carbon, nitrogen, oxygen, phosphorous, sulfur, fluorine and chlorine, such as ²H, ³H, ¹³C, ¹¹C, ¹⁴C, ¹⁵N, ¹⁸O, ¹⁷O, ³¹P, ³²P, ³⁵S, ¹⁸F, and ³⁶Cl, respectively. Compounds of the present invention, prodrugs thereof, and pharmaceutically acceptable salts of said compounds or of said prodrugs which contain the aforementioned isotopes and/or other isotopes of other atoms are within the scope of this invention. Certain isotopically labelled compounds of the present invention, for example those into which radioactive isotopes such as ³H, ¹⁸F, ¹¹C and ¹⁴C are incorporated, are useful in drug and/or substrate tissue distribution assays and others such as ¹¹C and ¹⁸F are useful for imaging studies i.e., PET, MRI, etc. Further, substitution with heavier isotopes such as deuterium, i.e., ²H, can afford certain therapeutic advantages resulting from greater metabolic stability, for example increased in vivo half-life or reduced dosage requirements and, hence, may be preferred in some circumstances. Isotopically labelled compounds of formula I of this invention and prodrugs thereof can generally be prepared by carrying out the procedures disclosed in the Schemes and/or in the Examples below, by substituting a readily available isotopically labelled reagent for a non-isotopically labelled reagent.

The term “alkyl”, as used herein, unless otherwise indicated, includes saturated monovalent hydrocarbon radicals having straight, branched or cyclic moieties or combinations thereof. Examples of “alkyl” groups include, but are not limited to, methyl, ethyl, propyl, isopropyl, butyl, iso- sec- and tert-butyl, pentyl, hexyl, heptyl, 3-ethylbutyl, cyclopropyl, cyclobutyl, cyclopentyl, cyclohexyl, cycloheptyl, norbornyl, and the like.

The term “heterocycle”, as used herein, unless otherwise indicated, includes 4, 5 or 6 membered ring containing at least one N, O, or S heteroatom which includes both aromatic and non-aromatic ring systems and includes fusion to a 5 or 6 membered aromatic, heteroaromatic, non-aromatic carbocycle, or non-aromatic heterocycle. Examples of heterocycles include, but are not limited to furan, tetrahydrofuran, thiophene, pyrrole, pyrrolidine, oxazole, thiazole, imidazole, pyrazole, isoxazole, isothiazole, triazole, tetrazole, pyran, pyridine, piperidine, morpholine, pyridazaine, pyrimidine, pyrazine, piperazine, and the fused bicyclic heterocycles: indolizine, indole, isoindole, indoline, benzofuran, benzothiophene, indazole, benzimidazole, benzthiazole, quinoline, isoquinoline, quinazoline, azetidine, oxetane, and ethylenedioxy benzene.

The term “one or more substituents”, as used herein, refers to a number of substituents that equals from one to the maximum number of substituents possible based on the number of available bonding sites.

The terms “halo” and “halogen”, as used herein, unless otherwise indicated, include, fluoro, chloro, bromo and iodo.

It will be noted that the structure of some of the compounds of this invention includes asymmetric carbon atoms. It is to be understood accordingly that the isomers arising from such asymmetry (e.g., all enantiomers and diastereomers) are included within the scope of this invention, unless indicated otherwise. Such isomers can be obtained in substantially pure form by classical separation techniques and by stereochemically controlled synthesis. Furthermore, the structures and other compounds and moieties discussed in this application also include all tautomers thereof.

The term “treating”, as used herein, refers to reversing, alleviating, inhibiting the progress of, or preventing the disorder or condition to which such term applies, or preventing one or more symptoms of such condition or disorder.

The term “treatment”, as used herein, refers to the act of treating, as “treating” is defined immediately above. The term “treatment” also includes the diminishment or alleviation of at least one symptom associated or-caused by the disorder being treated. For example, treatment can be diminishment of several symptoms of a disorder or complete eradication of a disorder.

The compounds of formula I and their pharmaceutically acceptable salts are also referred to herein, collectively, as the “novel compounds of this invention” and the “active compounds of this invention”.

Additional benefits and features of the present invention will become apparent to those skilled in the art from a review of the following detailed description, taken in conjunction with the example and the appended claims. It should be noted, however, that while the invention is susceptible of embodiments in various forms, described hereafter are specific preferred embodiments of the invention with the understanding that the present disclosure is intended as illustrative, and is not intended to limit the invention to the specific embodiments described herein.

### DETAILED DESCRIPTION OF THE INVENTION

The compounds of formula I of the present invention may be prepared as described in the following reaction scheme. Unless otherwise indicated, R¹-R⁸ in the reaction scheme and discussion that follow, are as defined above.

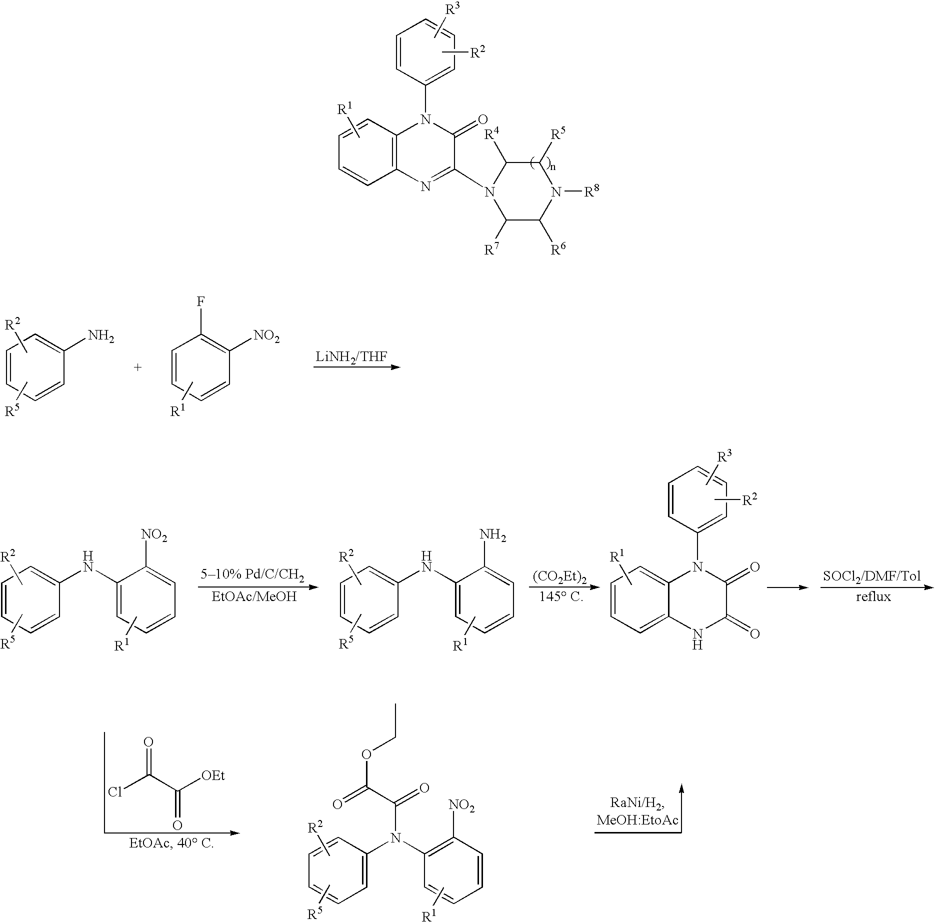

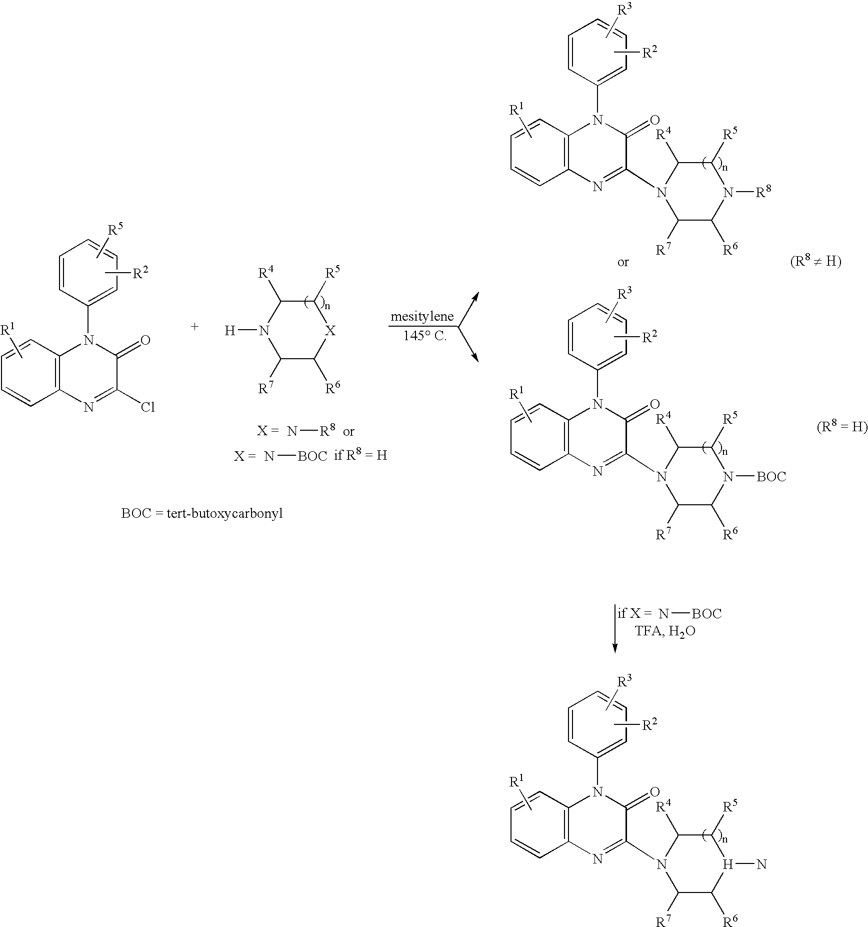

The compounds of the formula I and their pharmaceutically acceptable salts can be administered to mammals via either the oral, parenteral (such as subcutaneous, intravenous, intramuscular, intrasternal and infusion techniques), rectal, buccal or intranasal routes. In general, these compounds are most desirably administered in doses ranging from about 0.1 mg to about 1000 mg per day, in single or divided doses (I, from 1 to 4 doses per day), although variations will necessarily occur depending upon the species, weight and condition of the patient being treated and the patient's individual response to said medicament, as well as on the type of pharmaceutical formulation chosen and the time period and interval at which such administration is carried out. However, a dosage level that is in the range of about 25 mg to about 100 mg per day is most desirably employed. In some instances, dosage levels below the lower limit of the aforesaid range may be more than adequate, while in other cases still larger doses may be employed without causing any harmful side effects, provided that such higher dose levels are first divided into several small doses for administration throughout the day.

The compounds of the present invention may be administered alone or in combination with pharmaceutically acceptable carriers or diluents by any of the routes previously indicated, and such administration may be carried out in single or multiple doses. More particularly, the novel therapeutic agents of this invention can be administered in a wide variety of different dosage forms, i.e., they may be combined with various pharmaceutically acceptable inert carriers in the form of tablets, capsules, lozenges, troches, hard candies, suppositories, jellies, gels, pastes, ointments, aqueous suspensions, injectable solutions, elixirs, syrups, and the like. Such carriers include solid diluents or fillers, sterile aqueous media and various non-toxic organic solvents, etc. Moreover, oral pharmaceutical compositions can be suitably sweetened and/or flavored. In general, the weight ratio of the novel compounds of this invention to the pharmaceutically acceptable carrier will be in the range from about 1:6 to about 2:1, and preferably from about 1:4 to about 1:1.

For oral administration, tablets containing various excipients such as microcrystalline cellulose, sodium citrate, calcium carbonate, dicalcium phosphate and glycine may be employed along with various disintegrants such as starch (and preferably corn, potato or tapioca starch), alginic acid and certain complex silicates, together with granulation binders like polyvinylpyrrolidone, sucrose, gelatin and acacia. Additionally, lubricating agents such as magnesium stearate, sodium lauryl sulfate and talc are often very useful for tabletting purposes. Solid compositions of a similar type may also be employed as fillers in gelatin capsules; preferred materials in this connection also include lactose or milk sugar as well as high molecular weight polyethylene glycols. When aqueous suspensions and/or elixirs are desired for oral administration, the active ingredient may be combined with various sweetening or flavoring agents, coloring matter or dyes, and, if so desired, emulsifying and/or suspending agents as well, together with such diluents as water, ethanol, propylene glycol, glycerin and various like combinations thereof.

For parenteral administration, solutions of a compound of the present invention in either sesame or peanut oil or in aqueous propylene glycol may be employed. The aqueous solutions should be suitably buffered (preferably pH greater than 8) if necessary and the liquid diluent first rendered isotonic. These aqueous solutions are suitable for intravenous injection purposes. The oily solutions are suitable for intra-articular, intra-muscular and subcutaneous injection purposes. The preparation of all these solutions under sterile conditions is readily accomplished by standard pharmaceutical techniques well known to those skilled in the art.

This invention relates to methods of treating a central nervous system disorder or condition such as ADHD, anxiety, depression, schizophrenia and the other disorders referred to in the description of the methods of the present invention, wherein a novel compound of this invention and one or more of the other active agents referred to above (e.g., an NK1 receptor antagonist, an anxiolytic an antipsychotic agent, tricyclic antidepressant, 5HT1B receptor antagonist, or serotonin reuptake inhibitor) are administered together, as part of the same pharmaceutical composition, as well as to methods in which such active agents are administered separately as part of an appropriate dose regimen designed to obtain the benefits of the combination therapy. The appropriate dose regimen, the amount of each dose of an active agent administered, and the specific intervals between doses of each active agent will depend upon the subject being treated, the specific active agent being administered and the nature and severity of the specific disorder or condition being treated. In general, the novel compounds of this invention, when used as a single active agent or in combination with another active agent, will be administered to an adult human in an amount from about 3 mg to about 300 mg per day, in single or divided doses, preferably from about 25 to about 100 mg per day. Such compounds may be administered on a regimen of up to 6 times per day, preferably 1 to 4 times per day, especially 2 times per day and most especially once daily. Variations, may nevertheless occur depending upon the species of animal being treated and its individual response to said medicament, as well as on the type of pharmaceutical formulation chosen and the time period and interval at which such administration is carried out. In some instances, dosage levels below the lower limit of the aforesaid range may be more than adequate, while in other cases still larger doses may be employed without causing any harmful side effect, provided that such larger doses are first divided into several small doses for administration throughout the day.

A proposed daily dose of an atypical anti psychotic, preferably piprasidone, in the combination methods and compositions of this invention, for oral, parenteral or buccal administration to the average adult human for the treatment of the conditions referred to above, is from about 0.1 mg to about 2000 mg, preferably from about 1 mg to about 200 mg of an atypical anti psychotic per unit dose, which could be administered, for example, 1 to 4 times per day. A proposed daily dose of a 5HT1 B receptor antagonist in the combination methods and compositions of this invention, for oral, parenteral, rectal or buccal administration to the average adult human for the treatment of the conditions referred to above, is from about 0.01 mg to about 2000 mg, preferably from about 0.1 mg to about 200 mg of the 5HT1 B receptor antagonist per unit dose, which could be administered, for example, 1 to 4 times per day.

For intranasal administration or administration by inhalation, the novel compounds of the invention are conveniently delivered in the form of a solution or suspension from a pump spray container that is squeezed or pumped by the patient or as an aerosol spray presentation from a pressurized container or a nebulizer, with the use of a suitable propellant, e.g., dichlorodifluoromethane, trichlorofluoromethane, dichlorotetrafluoroethane, carbon dioxide or other suitable gas. In the case of a pressurized aerosol, the dosage unit may be determined by providing a valve to deliver a metered amount. The pressurized container or nebulizer may contain a solution or suspension of the active compound. Capsules and cartridges (made, for example, from gelatin) for use in an inhaler or insufflator may be formulated containing a powder mix of a compound of the invention and a suitable powder base such as lactose or starch. Formulations of the active compounds of this invention for treatment of the conditions referred to above in the average adult human are preferably arranged so that each metered dose or “puff” of aerosol contains 20 μg to 1000 μg of active compound. The overall daily dose with an aerosol will be within the range 100 μg to 10 mg. Administration may be several times daily, for example 2, 3, 4 or 8 times, giving for example, 1, 2 or 3 doses each time.

The ability of the compounds of this invention to bind to the hNET and serotonin SERT receptor can be determined using conventional radioligand receptor binding assays. The receptors can be heterologously expressed in cell lines and experiments conducted in membrane preparations from the cell lines using procedures outlined below. IC₅₀ concentrations can be determined by nonlinear regression of concentration-dependent reduction in specific binding. The Cheng-Prussoff equation can be used to convert the IC₅₀ to Ki concentrations.

hNET Receptor Binding:

Cell pastes of HEK-293 cells transfected with the human norepinephrine transporter were supplied by the Pfizer Ann Arbor Protein Expression and Production group. Pellets were resuspended in 400 to 700 ml of Krebs-HEPES assay buffer (25 mM HEPES, 122 mM NaCl, 3 mM KCl, 1.2 mM MgSO₄, 1.3 mM CaCl₂, and 11 mM glucose, pH 7.4) with a Polytron homogenizer at setting 7 for 30 sec. Aliquots of membranes (5 mg/ml protein) were stored in liquid nitrogen until used.

The binding assay was set up in Beckman deep-well polypropylene plates with a total volume of 250 μl containing: drug (10⁻⁵M to 10⁻¹²M), cell membranes, and 50 pM [¹²⁵I]-RTI-55 (Perkin Elmer, NEX-272; specific activity 2200 Ci/mmol). The reaction was incubated by gentle agitation for 90 min at room temperature and was terminated by filtration through Whatman GF/C filter plates using a Brandel 96-well plate harvester. Scintillation fluid (100 μl) was added to each well, and bound [¹²⁵I]-RTI-55 was determined using a Wallac Trilux Beta Plate Counter. Test compounds were run in duplicate, and specific binding was defined as the difference between binding in the presence and absence of 10 μM desipramine.

Excel and GraphPad Prism software were used for data calculation and analysis. IC₅₀ values were converted to Kᵢ values using the Cheng-Prusoff equation.

hSERT Receptor Binding:

Cell pastes of HEK-293 cells transfected with the human seritonin transporter were supplied by the Pfizer Ann Arbor Protein Expression and Production group. Pellets were resuspended in 400 to 700 ml of Krebs-HEPES assay buffer (25 mM HEPES, 122 mM NaCl, 3 mM KCl, 1.2 mM MgSO₄, 1.3 mM CaCl₂, and 11 mM glucose, pH 7.4) with a Polytron homogenizer at setting 7 for 30 sec. Aliquots of membranes (5 mg/ml protein) were stored in liquid nitrogen until used.

The binding assay was set up in Beckman deep-well polypropylene plates with a total volume of 250 μl containing: drug (10⁻⁵M to 10⁻¹²M), cell membranes, and 50 pM [¹²⁵I]-RTI-55 (Perkin Elmer, NEX-272; specific activity 2200 Ci/mmol). The reaction was incubated by gentle agitation for 90 min at room temperature and was terminated by filtration through Whatman GF/C filter plates using a Brandel 96-well plate harvester. Scintillation fluid (100 μl) was added to each well, and bound [¹²⁵I]-RTI-55 was determined using a Wallac Trilux Beta Plate Counter. Test compounds were run in duplicate, and specific binding was defined as the difference between binding in the presence and absence of 10 μM citalopram.

Excel and GraphPad Prism software were used for data calculation and analysis. IC₅₀ values were converted to Kᵢ values using the Cheng-Prusoff equation.

The hNET activities of compounds of the present invention are set forth in Table 1.

TABLE 1

| Ex- | | hNET Ki | | | |
|---|---|---|---|---|---|
| ample # | Structure | (nM) | MP (° C.) | MF | CHN |
| 1 | 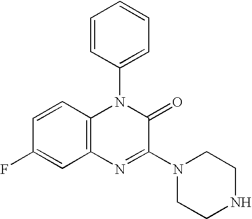 | 12 | 150-152 | C₁₈H₁₇FN₄O.0.05 C₄H₈O₂ | C, 66.18; H, 5.24; N, 16.93; F, 5.80 |
| | 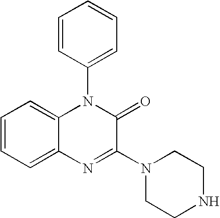 | 21 | 290-292 | C₁₈H₁₈N₄O.1.0 HCl.0.15 H₂O.0.03 C₄H₈O₂ | C, 62.52; H, 5.47; N, 16.10; Cl, 10.38 |
| 3 | 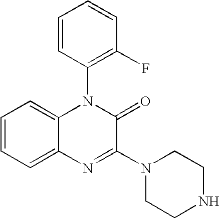 | 11 | 261-263 | C₁₈H₁₇FN₄O.1.0 HCl.0.4 H₂O | C, 58.73; H, 5.06; N, 14.88; Cl, 9.70; F, 5.23 |
| | 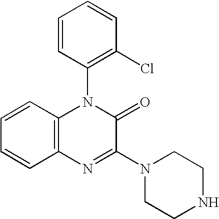 | 44 | 135-137 | C₁₈H₁₇ClN₄O.0.03 C₄H₁₀O | C, 63.23; H, 4.92; N, 16.07; Cl, 10.28 |
| | 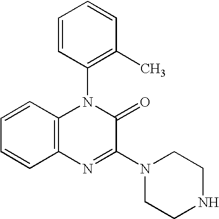 | 69 | 151-153 | C₁₉H₂₀N₄O.0.06 C₄H₈O₂.0.02 C₄H₁₀O | C, 70.84; H, 6.30; N, 17.12 |
| 6 | 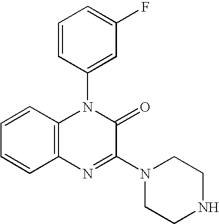 | 38 | 149-150 | C₁₈H₁₇FN₄O.0.02 C₄H₈O₂ | C, 66.77; H, 5.10; N, 17.08; F, 5.87 |
| | 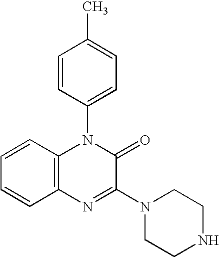 | 36 | 197-199 | C₁₉H₂₀N₄O.C₄H₄O₄ | C, 63.23; H, 5.45; N, 12.74 |
| | 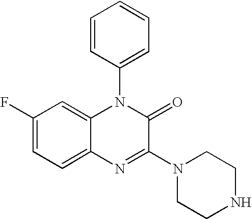 | 59 | 142-143 | C₁₈H₁₇FN₄O | C, 66.47; H, 4.94; N, 17.05; F, 5.71 |
| 9 | 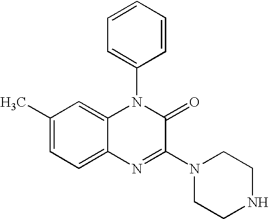 | 82 | 166-168 | C₁₉H₂₀N₄O.0.09 C₄H₁₀O | C, 70.71; H, 6.37; N, 17.37 |
| 10 | 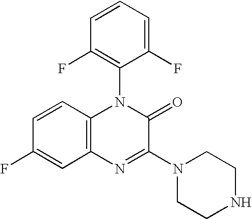 | 15 | 146-147 | C₁₈H₁₅F₃N₄O.0.03 C₄H₈O₂ | C; 59.93; H, 4.15; N, 15.29; F, 15.95 |
| 11 | 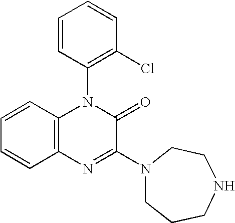 | 41 | 159-161 | C₁₉H₁₉ClN₄O.C₄H₄O₄ | C, 58.63; H, 4.84; N, 11.85; Cl, 7.48 |
| 12 | 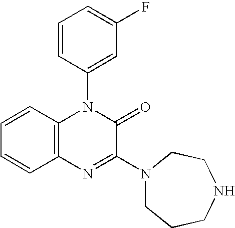 | 60 | 128-129 | C₁₉H₁₉FN₄O.0.06 C₄H₈O₂ | C, 67.18; H, 5.68; N, 16.17; F, 5.41 |
| 13 | 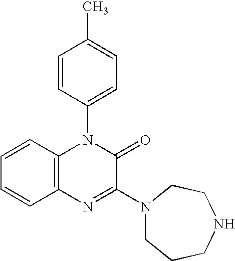 | 97 | 199-200 | C₂₀H₂₂N₄O.C₄H₄O₄ | C, 63.83; H, 5.79; N, 12.21 |
| 14 | 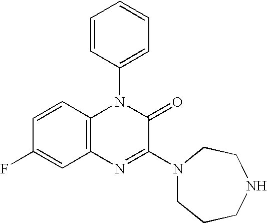 | 27 | 151-152 | C₁₉H₁₉FN₄O.0.07C₄H₈0₂.0.12H₂O | C, 66.42; H, 5.62; H, 16.02; F, 5.55 |
| 15 | 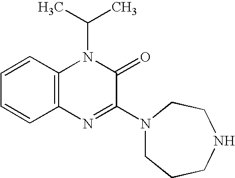 | 70 | 147-149 | C₁₈H₂₂N₄O.C₄H₄O₄ | C, 59.46; H, 6.53; N, 13.87 |

### EXAMPLE 1

### 6-Fluoro-1-phenyl-3-piperazin-1-yl-1H-quinoxalin-2-one

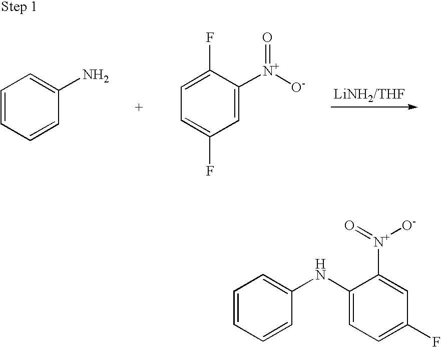

The aniline (3.36 ml, 36.88 mmol), 2,5-difluoronitrobenzene (4.00 ml, 36.88 mmol), and dry THF (37 ml) were combined and cooled in an ice-water bath. Lithium amide (2.12 g, 92.2 mmol) was added to the light orange solution very slowly, and the deep purple colored mixture stirred for 90 minutes. The reaction was quenched with 3N HCl until the aqueous layer was pH=1. The layers were separated, and the aqueous layer extracted with EtOAc (2×50 ml). The combined organics were wash with brine (50 ml), dried over MgSO₄, filter and concentrated. The dark orange solids were filtered through a plug of silica eluting with 20% CH₂Cl₂/hexanes to give the desired product, 5.012 g (58%).

¹H NMR (400 MHz, CDCl₃) δ ppm 7.19 (m, 5 H) 7.42 (m, 2 H) 7.91 (dd, J=9.03, 2.93 Hz, 1 H) 9.34 (s, 1 H) MS (APCl+): 233 (M+1, 100%); (APCl−): 231 (M−1, 100%), 232 (M, 45%)

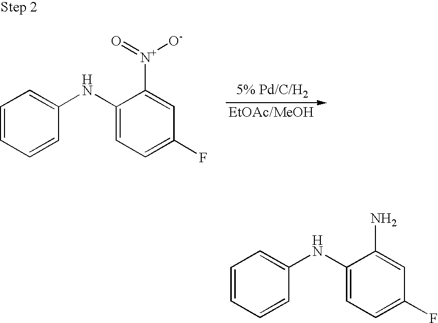

(4-fluoro-2-nitro-phenyl)-phenyl-amine (5.012 g, 21.5 mmol) was dissolved in EtOAc/MeOH (50 ml/50 ml), and charged with 5% Pd/C (0.50 g). The suspension was purged with hydrogen, and the reaction stirred at RT under hydrogen atmosphere (1 atm) for 18 hrs. The catalyst was filtered off and washed with EtOAc. The filtrate was concentrated and the material chromatographed on silica eluting with 30% CH₂Cl₂/hexanes to give 3.285 g (75%) of desired product.

¹H NMR (400 MHz, CDCl₃) δ ppm 3.95 (s, 2 H) 4.97 (s, 1 H) 6.42 (td, J=8.43, 2.69 Hz, 1 H) 6.49 (dd, J=10.14, 2.81 Hz, 1 H) 6.64 (d, J=7.81 Hz, 2 H) 6.79 (t, J=7.33 Hz, 1 H) 7.02 (dd, J=8.55, 5.86 Hz, 1 H) 7.18 (m, 2 H).

MS (APCl+): 203 (M+1, 100%); (APCl−): 201 (M−1, 100%)

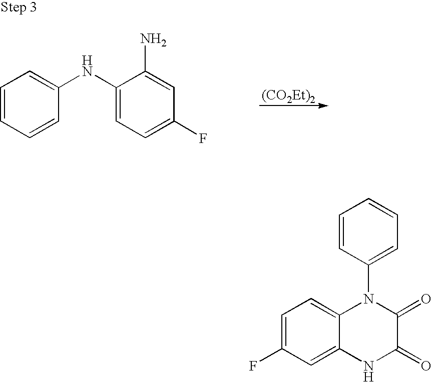

4-fluoro-N-phenyl-benzene-1,2-diamine (3.285 g, 16.24 mmol) was combined with diethyl oxalate (6.6 ml, 48.73 mmol) and heated to 145° C. for 18 hrs under N₂ atmosphere. The gray suspension was cooled to RT, and ether (40 ml) added. The solids were filtered, washed with ether and air-dried to give 3.528 g (84%) of desired product.

¹H NMR (400 MHz, DMSO-D6) δ ppm 6.26 (dd, J=9.28, 5.13 Hz, 1 H) 6.82 (td, J=8.79, 2.93 Hz, 1 H) 6.97 (dd, J=9.28, 2.69 Hz, 1 H) 7.36 (m, 2 H) 7.52 (m, 1 H) 7.60 (m, 2 H) 12.17 (s, 1 H)

MS (APCl+): 257 (M+1, 100%); (APCl−): 255 (M−1, 100%).

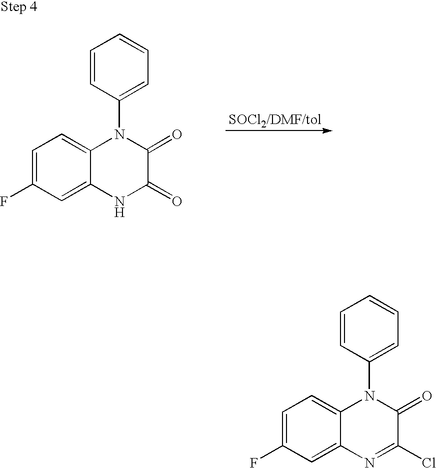

6-fluoro-1-phenyl-1,4-dihydro-quinoxaline-2,3-dione (2.010 g, 7.84 mmol) was combined with anhydrous DMF (5.5 ml) and anhydrous toluene (78 ml). Thionyl chloride (0.89 ml, 12.16 mmol) was added, and the suspension heated to reflux under N₂ for three hrs. The solvent was evaporated, the crude material washed with EtOAc and concentrated to dryness. The crude material was purified by flash chromatography by eluting with CH₂Cl₂ to give 1.86 g (86%) of product.

¹H NMR (400 MHz, CDCl₃) δ ppm 6.66 (dd, J=9.27, 4.88 Hz, 1 H) 7.14 (ddd, J=9.27, 7.81, 2.93 Hz, 1 H) 7.28 (ddd, J=6.59, 1.71, 1.46 Hz, 2 H) 7.55 (dd, J=8.42, 3.05 Hz, 1 H) 7.62 (m, 3 H) MS (APCl+): 275 (M+1, 100%), 277 (M+3, 35%); (APCl−):274 (M−, 100%)

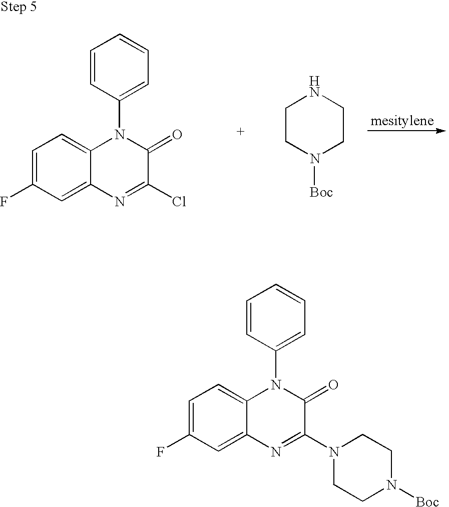

3-chloro-6-fluoro-1-phenyl-1H-quinoxalin-2-one (0.398 g, 1.449 mmol) was combined with piperazine-1-carboxylic acid tert-butyl ester (1.08 g, 5.8 mmol) and mesitylene (5 ml). The mixture was heated to 145° C. for five hours. The reaction was partitioned between water/EtOAc (15 ml/15 ml), and the organics separated. The aqueous layer was extracted with EtOAc (3×25 ml). The combined organics were washed with brine (25 ml), dried over Na₂SO₄, filtered, and concentrated. The crude material was purified by flash chromatography on silica eluting with CH₂Cl₂ for 5 minutes, then with 35% EtOAc/hexanes to give 0.568 g (92%) of desired product.

¹H NMR (400 MHz, CDCl₃) δ ppm 1.48 (s, 9 H) 3.57 (m, 4 H) 4.02 (m, 4 H) 6.43 (dd, J=9.27, 5.12 Hz, 1 H) 6.77 (ddd, J=9.15, 8.05, 2.81 Hz, 1 H) 7.27 (m, 3 H) 7.55 (m, 1 H) 7.60 (m, 2 H) MS (APCl+): 425 (M+1, 50%), 325 (M−99, -BOC, 100%)

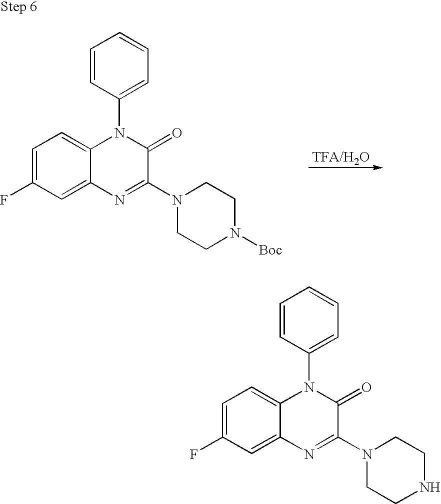

4-(7-fluoro-3-oxo-4-phenyl-3,4-dihydro-quinoxalin-2-yl)-piperazine-1-carboxylic acid tert-butyl ester (0.562 g, 1.324 mmol) was combined with trifluoroacetic acid (2.65 ml) and water (0.26 ml). The reaction was stirred at RT for 1 hr. The reaction was diluted with MeOH (5 ml) and passed through a 10 g Varian Mega Bond Elut SCX column prewashed with 5% AcOH/MeOH. The column was eluted first with MeOH until washings were pH neutral, then with 1.0M NH₃/MeOH to obtain product. The filtrate was concentrated, and the product washed with EtOAc and concentrated to dryness (3×). The solids were dissolved in EtOAc, filter through a plug of Celite, and concentrated to dryness. The solids were washed with hexanes/ether, filtered and dried under vacuum at 65° C. to give 0.347 g (80%) of desired product.

MP=150-152° C.

Analysis for C₁₈H₁₇FN₄O.0.05 C₄H₈O₂, Calcd: C, 66.49; H, 5.33; N, 17.04; F, 5.78. Found: C, 66.18; H, 5.24; N, 16.93; F, 5.80.

¹H NMR (400 MHz, CDCl₃) δ ppm 1.72 (s, 1 H) 3.00 (m, 4 H) 4.01 (m, 4 H) 6.40 (dd, J=9.03, 5.12 Hz, 1 H) 6.74 (ddd, J=9.15, 8.05, 2.81 Hz, 1 H) 7.24 (m, 3 H) 7.57 (m, 3 H)

HPLC: 97.70%, 4.647 min, 214 nM, Phenomenex Luna 5u C18, 4.6×150 mm, 65:35 (H2O/MeCN)+0.1%TFA, 1 ml/min

### EXAMPLE 2

### 1-Phenyl-3-piperazin-1-yl-1H-quinoxalin-2-one Hydrochloride

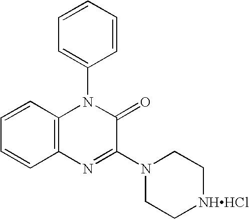

Following the procedures set forth in Example 1, but substituting (2-nitro-phenyl)-phenyl-amine for (4-fluoro-2-nitro-phenyl)-phenyl-amine in step 2, afforded the parent compound. The HCl salt was obtained by treating a 0.03M solution of the parent in EtOAc with 1 equivalent of a 1 N solution of HCl in ether in a 92% yield.

MP=290−292° C. Analysis for C₁₈H₁₈N₄O.1.0 HCl.0.15 H₂O.0.03 C₄H₈O₂: C, 62.51; H, 5.66; N, 16.09; Cl, 10.18. Found: C, 62.52; H, 5.47; N, 16.10; Cl, 10.38.

### EXAMPLE 3

### 1-(2-Fluoro-phenyl)-3-piperazin-1-yl-1H-quinoxalin-2-one Hydrochloride

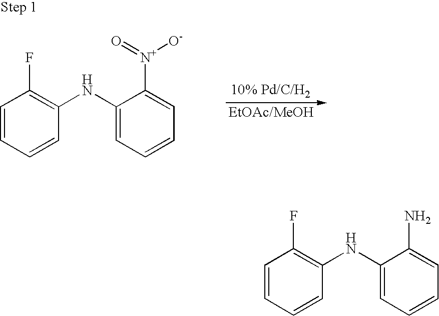

(2-fluoro-phenyl)-(2-nitro-phenyl)-amine (5.012 g, 21.5 mmol) was dissolved in EtOAc/MeOH (300 ml/110 ml), and charged with 10% Pd/C (0.50 g). The suspension was purged with hydrogen, and the reaction stirred at RT under hydrogen atmosphere (1 atm) for 4 hrs. The catalyst was filtered off and washed with EtOAc. The filtrate was concentrated to give 4.35 g (99%) of desired product.

¹H NMR (400 MHz, CDCl₃) δ ppm 3.80 (s, 2 H) 5.35 (s, 1 H) 6.66 (m, 1 H) 6.73 (m, 2 H) 6.81 (dd, J=7.94, 1.34 Hz, 1 H) 6.93 (t, J=7.69 Hz, 1 H) 7.05 (dd, 2 H) 7.11 (dd, J=7.82, 1.47 Hz, 1 H) MS (APCl+): 203 (M+1, 100%)

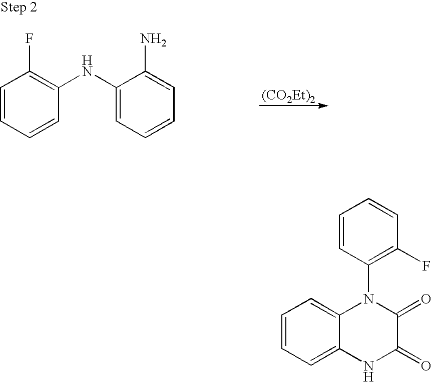

N-(2-fluoro-phenyl)-benzene-1,2-diamine (4.26 g, 21.06 mmol) was combined with diethyl oxalate (11.4 ml, 84.26 mmol) and heated to 145° C. for 18 hrs under N₂ atmosphere. The gray suspension was cooled to RT, and ethanol (40 ml) added. The solids were filtered, washed with ethanol (2×5 ml), and air-dried to give 4.67 g (86%) of desired product.

¹H NMR (400 MHz, DMSO-D6) δ ppm 6.38 (d, J=8.06 Hz, 1 H) 7.01 (td, J=7.82, 1.47 Hz, 1 H) 7.16 (td, J=7.69, 1.22 Hz, 1 H) 7.23 (m, 1 H) 7.44 (m, 1 H) 7.52 (m, 2 H) 7.62 (m, 1 H) 12.24 (s, 1 H)

MS (APCl+): 257 (M+1, 100%)

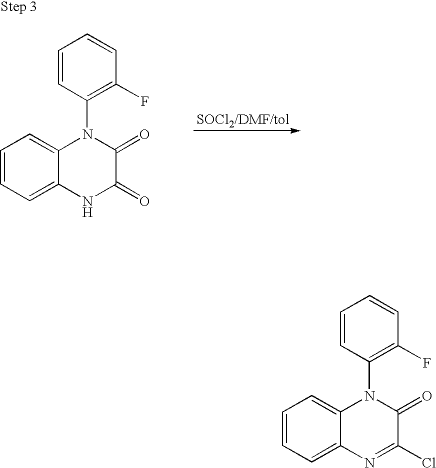

1-(2-fluoro-phenyl)-1,4-dihydro-quinoxaline-2,3-dione (2.016 g, 7.87 mmol) was combined with anhydrous DMF (5.2 ml) and anhydrous toluene (75 ml). Thionyl chloride (0.9 ml, 12.34 mmol) was added, and the suspension heated to reflux under N₂ for three hrs. The reaction was quenched with water (40 ml), the organics separated, dried over MgSO₄, filtered and concentrated. The crude material was purified by flash chromatography on silica by first dissolving the product in CH₂Cl₂, and then eluting with 25% EtOAc/hexanes to give 1.26 g (58%) of product.

¹H NMR (400 MHz, CDCl₃) δ ppm 6.71 (m, 1 H) 7.38 (m, 5 H) 7.59 (m, J=7.81, 7.81, 5.12, 1.95 Hz, 1 H) 7.87 (dd, J=7.93, 1.34 Hz, 1 H)

MS (APCl+): 275 (M+1, 100%), 277 (M+3, 80%)

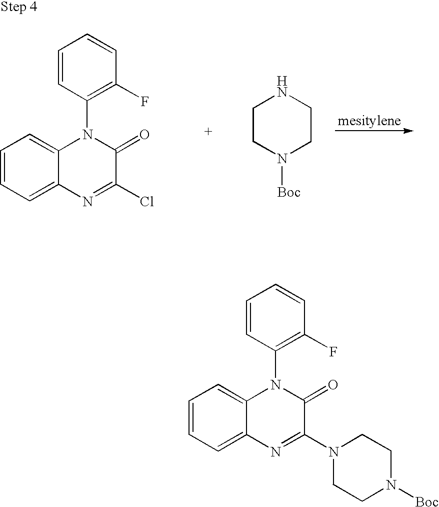

3-chloro-1-(2-fluoro-phenyl)-1H-quinoxalin-2-one (1.254 g, 4.565 mmol) was combined with piperazine-1-carboxylic acid tert-butyl ester (3.40 g, 22 mmol) and mesitylene (6 ml). The mixture was heated to 140° C. for 90 minutes. The reaction was partitioned between water/EtOAc (50 ml/50 ml), and the organics separated. The aqueous layer was extracted with EtOAc (3×25 ml). The combined organics were washed with brine (25 ml), dried over Na₂SO₄, filtered, and concentrated. The crude material was purified by flash chromatography on silica eluting with CH₂Cl₂ for 5 minutes, then with 30% EtOAc/hexanes to give 1.86 g (96%) of desired product.

¹H NMR (400 MHz, CDCl₃) δ ppm 1.48 (s, 9 H) 3.57 (m, 4 H) 3.96 (m, 4 H) 6.54 (d, J=8.30 Hz, 1 H) 7.09 (ddd, J=8.42, 7.20, 1.46 Hz, 1 H) 7.23 (td, J=7.69, 1.22 Hz, 1 H) 7.34 (m, 3 H) 7.54 (m, 1 H) 7.58 (dd, J=8.06, 1.46 Hz, 1 H)

MS (APCl+): 425 (M+1, 80%), 325 (M−99, -BOC, 100%)

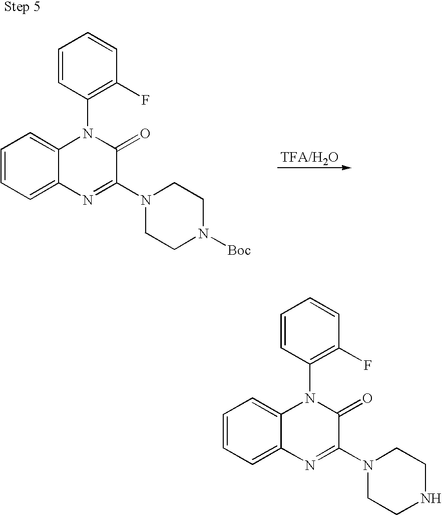

4-[4-(2-fluoro-phenyl)-3-oxo-3,4-dihydro-quinoxalin-2-yl]-piperazine-1-carboxylic acid tert-butyl ester (1.137 g, 2.679 mmol) was combined with trifluoroacetic acid (14 ml) and water (1.4 ml). The reaction was stirred at RT for 1 hr. The reaction was divided into three equal parts by volume, and each part passed through a 10 g Varian Mega Bond Elut SCX column prewashed with 5% AcOH/MeOH. The column was eluted first with MeOH until washings were pH neutral, then with 1.0M NH₃/MeOH to obtain product. The filtrate was concentrated, and the product washed with EtOAc and concentrated to dryness (3×). The solids were dissolved in EtOAc, filter through a plug of Celite, and concentrated to dryness. The solids were washed with ether, filtered and air dried to give 1.380 g (60%) of desired product.

¹H NMR (400 MHz, CDCl₃) δ ppm 3.02 (m, 4 H) 3.97 (m, 4 H) 6.52 (d, J=8.30 Hz, 1 H) 7.07 (td, J=7.81, 1.46 Hz, 1 H) 7.21 (td, J=7.69, 1.22 Hz, 1 H) 7.34 (m, 3 H) 7.53 (m, 1 H) 7.57 (dd, J=8.05, 1.46 Hz, 1 H) MS (APCl+): 325 (M+1, 100%)

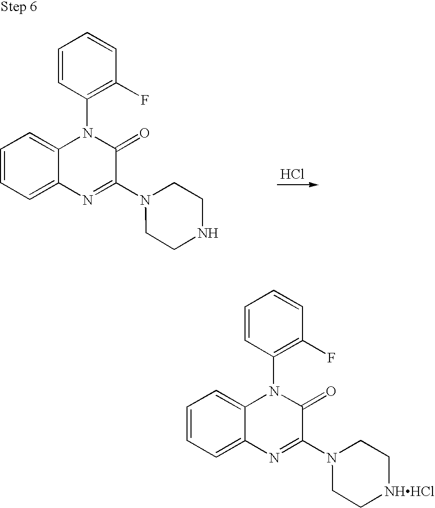

1-(2-fluoro-phenyl)-3-piperazin-1-yl-1H-quinoxalin-2-one (0.425 g, 1.310 mmol) was dissolved in EtOAc (60 ml). 1N HCl/ether (1.3 ml) was added dropwise to the solution, and the suspension stirred at RT 15 minutes. Methanol (5 ml) was added, and the mixture stirred at RT for 45 minutes. The solids were filtered, and dried under vacuum at 95° C. overnight to give 0.2518 g (53%) of desired product.

MP=261-263° C. Analysis for C₁₈H₁₇FN₄O.1.0 HCl.0.40 H₂O: C, 58.74; H, 5.15; N, 15.22; Cl, 9.63; F, 5.16. Found: C, 58.73; H, 5.06; N, 14.88; Cl, 9.70; F, 5.23.

1H NMR (400 MHz, DMSO-D6) δ ppm 3.22 (s, 4 H) 4.07 (m, 4 H) 6.45 (d, J=8.06 Hz, 1 H) 7.17 (m, 1 H) 7.26 (m, 1 H) 7.46 (m, J=8.06 Hz, 1 H) 7.52 (m, 3 H) 7.64 (m, 1 H) 9.17 (s, 2 H)

### EXAMPLE 4

### 1-(2-Chloro-phenyl)-3-piperazin-1-yl-1H-quinoxalin-2-one

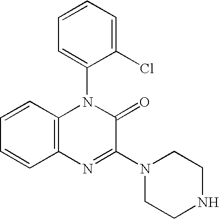

Following the procedures set forth in Example 1, but substituting (2-chloro-phenyl)-(2-nitro-phenyl)-amine for (4-fluoro-2-nitro-phenyl)-phenyl-amine in step 2, afforded the title compound in similar yield.

MP=135-137° C. Analysis for C₁₈H₁₇ClN₄O.0.03 C₄H₁₀O: C, 63.44; H, 5.08; N, 16.33; Cl, 10.34. Found: C, 63.23; H, 4.92; N, 16.07; Cl,10.28.

### EXAMPLE 5

### 3-Piperazin-1-yl-1-o-tolyl-1H-quinoxalin-2-one

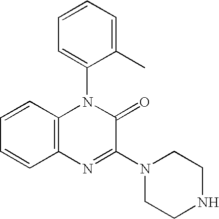

Following the procedures set forth in Example 1, but substituting (2-nitro-phenyl)-o-tolyl-amine for (4-fluoro-2-nitro-phenyl)-phenyl-amine in step 2, afforded the title compound in similar yield.

MP=151-153° C. Analysis for C₁₉H₂₀N₄O.0.06 C₄H₈O₂.0.02 C₄H₁₀O: C, 70.93; H, 6.37; N, 17.13. Found: C, 70.84; H, 6.30; N, 17.12.

### EXAMPLE 6

### 1-(3-Fluoro-phenyl)-3-piperazin-1-yl-1H-quinoxalin-2-one

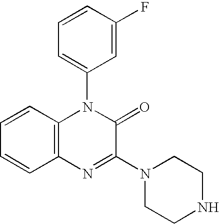

Following the procedures set forth in Example 1, but substituting (3-fluoro-phenyl)-(2-nitro-phenyl)-amine for (4-fluoro-2-nitro-phenyl)-phenyl-amine in step 2, afforded the title compound in similar yield.

MP=149-150° C. Analysis for C₁₈H₁₇FN₄O.0.02 C₄H₈₂: C, 66.59; H, 5.30; N, 17.18; F, 5.83. Found: C, 66.77; H, 5.10; N, 17.08; F, 5.87.

### EXAMPLE 7

### 3-Piperazin-1-yl-1-p-tolyl-1H-quinoxalin-2-one Maleate

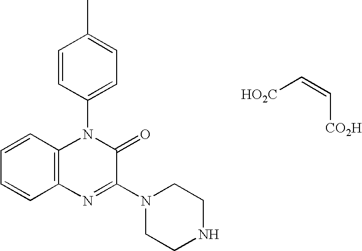

Following the procedures set forth in Example 1, but substituting (2-nitro-phenyl)-p-tolyl-amine for (4-fluoro-2-nitro-phenyl)-phenyl-amine in step 2, afforded the parent compound in similar yield. The maleate salt was obtained by treating a 0.10M solution of the parent compound in EtOAc with 1 equivalent of maleic acid in methanol (1 ml) in a 94% yield.

MP=197-199° C. Analysis for C₁₉H₂₀N₄O.C₄H₄O₄: C, 63.29; H, 5.54; N, 12.84. Found: C, 63.23; H, 5.45; N, 12.74.

### EXAMPLE 8

### 7-Fluoro-1-phenyl-3-piperazin-1-yl-1H-quinoxalin-2-one

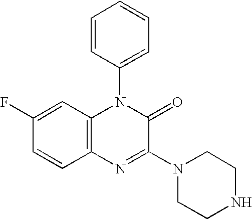

Following the procedures set forth in Example 1, but substituting (5-fluoro-2-nitro-phenyl)-phenyl-amine for (4-fluoro-2-nitro-phenyl)-phenyl-amine in step 2, afforded the title compound in similar yield.

MP=142-143° C. Analysis for C₁₈H₁₇FN₄O: C, 66.65; H, 5.28; N, 17.27; F, 5.86. Found: C, 66.47; H, 4.94; N, 17.05; F, 5.71.

### EXAMPLE 9

### 7-Methyl-1-phenyl-3-piperazin-1-yl-1H-quinoxalin-2-one

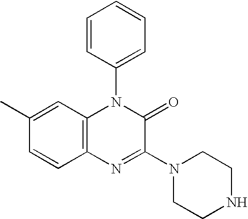

Following the procedures set forth in Example 1, but substituting (5-methyl-2-nitro-phenyl)-phenyl-amine for (4-fluoro-2-nitro-phenyl)-phenyl-amine in step 2, afforded the title compound in similar yield.

MP=166-168° C. Analysis for C₁₉H₂₀N₄O.0.09 C₄H₁₀O: C, 71.10; H, 6.44; N, 17.13. Found: C, 70.71; H, 6.37; N, 17.37.

### EXAMPLE 10

### 1-(2,6-Difluoro-phenyl)-6-fluoro-3-piperazin-1-yl-1H-quinoxalin-2-one

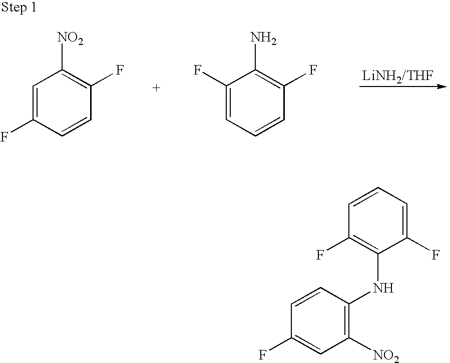

The 2,6-difluoroaniline, (3.97 ml, 36.88 mmol), 2,5-difluoronitrobenzene (4.00 ml g, 36.88 mmol), and dry THF (40 ml) were combined and cooled in an ice-water bath. Lithium amide (2.12 g, 92.2 mmol) was added to the light orange solution very slowly, and the deep purple colored mixture stirred for 2 hours. The reaction was quenched with 3N HCl (20 ml) and acidified with conc HCl until the aqueous layer was pH=1. The layers were separated, and the aqueous layer extracted with EtOAc (2×50 ml). The combined organics were wash with brine (50 ml), dried over MgSO₄, filter and concentrated. The dark orange solids were filtered through a plug of silica eluting with 40% CH₂Cl₂/hexanes to give the desired product, 9.054 g (92%).

¹H NMR (400 MHz, CDCl₃) δ ppm 6.71 (ddd, J=7.01, 4.58, 2.32 Hz, 1 H) 7.04 (t, J=7.93 Hz, 2 H) 7.23 (m, 2 H) 7.94 (dd, J=8.91, 2.81 Hz, 1 H) 8.88 (s, 1 H) MS (APCl−): 268 (M−, 100%)

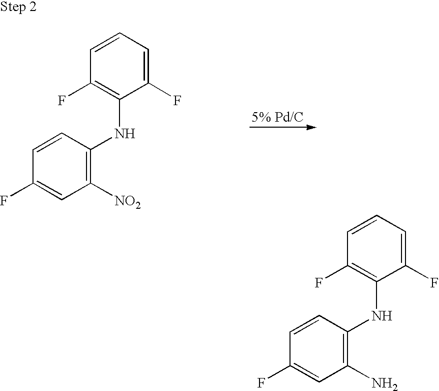

(2,6-Difluoro-phenyl)-(4-fluoro-2-nitro-phenyl)-amine (4.006 g, 14.9 mmol) was dissolved in EtOAc/MeOH (50 ml/50 ml), and charged with 5% Pd/C (0.40 g). The suspension was purged with hydrogen, and the reaction stirred at RT under hydrogen atmosphere (1 atm) for 18 hrs. The catalyst was filtered off and washed with EtOAc. The filtrate was concentrated and the material chromatographed on silica eluting with 15% EtOAc/hexanes to give 3.262 g (92%) of desired product.

¹H NMR (400 MHz, CDCl₃) δ ppm 3.99 (s, 2 H) 4.84 (s, 1 H) 6.37 (td, J=8.48, 2.81 Hz, 1 H) 6.49 (dd, J=9.88, 2.81 Hz, 1 H) 6.74 (m, 1 H) 6.88 (m, 3 H)

MS (APCl+): 239 (M+1, 100%); (APCl−): 237 (M−1, 100%)

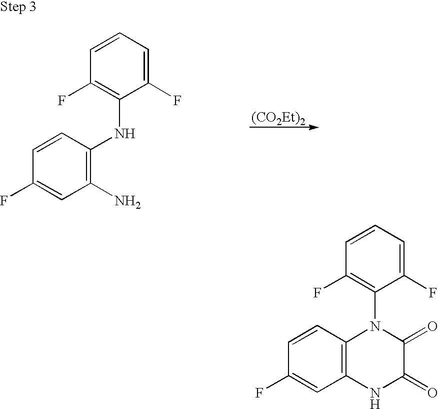

N¹-(2,6-Difluoro-phenyl)-4-fluoro-benzene-1,2-diamine (3.262 g, 13.69 mmol) was combined with diethyl oxalate (7.4 ml, 41 mmol) and heated to 150° C. for 18 hrs under N₂ atmosphere. NaH (1.10 g, 60% w/w, 27.4 mmol) was added to the reaction, and heated at 150° C. for 2 hours. The reaction was quenched with 1 N HCl, and extracted with EtOAc (3×50 ml). The combined organics were washed with brine, dried over MgSO₄, filtered and passed through a plug of silica eluting with EtOAc. The filtrate was concentrated to give a green oil. Ether (40 ml) was added and the mixture stirred at RT for 18 hours. The blue-green solids were filtered and air dried to give 2.545 g (63%) of desired product.

¹H NMR (400 MHz, DMSO-D6) δ ppm 6.59 (dd, J=9.03, 4.88 Hz, 1 H) 6.91 (td, J=8.72, 2.81 Hz, 1 H) 7.05 (dd, J=9.15, 2.81 Hz, 1 H) 7.45 (t, J=8.42 Hz, 2 H) 7.73 (m, J=8.60, 8.60, 6.47, 6.34 Hz, 1 H) 12.41 (s, 1 H)

MS (APCl+): 293 (M+1, 100%); (APCl−): 291 (M−1, 100%)

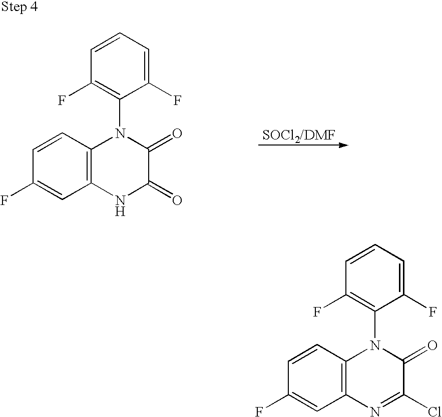

1-(2,6-difluoro-phenyl)-6-fluoro-1,4-dihydro-quinoxaline-2,3-dione (1.000 g, 3.422 mmol) was combined with anhydrous DMF (2.5 ml) and anhydrous toluene (35 ml). Thionyl chloride (0.39 ml, 5.3 mmol) was added, and the suspension heated to reflux under N₂ for three hrs. The solvent was evaporated, the crude material washed with EtOAc and concentrated to dryness. The crude material was purified by flash chromatography on silica by eluting with CH₂Cl₂ to give 0.782 g (74%) of product.

¹H NMR (400 MHz, CDCl₃) δ ppm 6.70 (dd, J=9.27, 4.64 Hz, 1 H) 7.20 (m, 3 H) 7.59 (m, 2 H) MS (APCl+): 311 (M+1, 100%), 313 (M+3, 50%); (APCl−): 310 (M−1, 100%)

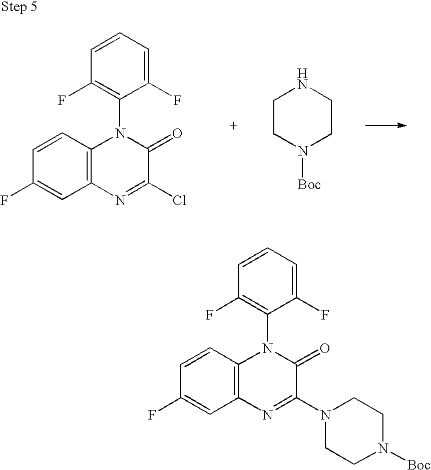

3-chloro-1-(2,6-difluoro-phenyl)-6-fluoro-1H-quinoxalin-2-one (0.350 g, 1.127 mmol) was combined with piperazine-1-carboxylic acid tert-butyl ester (0.839 g, 4.50 mmol) and mesitylene (5 ml). The mixture was heated to 150° C. for five hours. The reaction was partitioned between water/EtOAc (15 ml/15 ml), and the organics separated. The aqueous layer was extracted with EtOAc (3×25 ml). The combined organics were washed with brine (25 ml), dried over Na₂SO₄, filtered, and concentrated. The crude material was purified by flash chromatography on silica eluting with CH₂Cl₂ for 5 minutes, then with 25% EtOAc/hexanes to give 0.522 g (99%) of desired product.

¹H NMR (400 MHz, CDCl₃) 6 ppm 1.48 (s, 9 H) 3.57 (m, 4 H) 4.01 (m, 4 H) 6.48 (dd, J=9.03, 5.12 Hz, 1 H) 6.83 (ddd, J=9.03, 7.93, 2.81 Hz, 1 H) 7.16 (m, 2 H) 7.26 (dd, J=9.52, 2.68 Hz, 1 H) 7.53 (m, 1 H) MS (APCl+): 461 (M+1, 90%), 361 (M−99, -BOC, 100%); (APCl−): 460 (M−1, 100%)

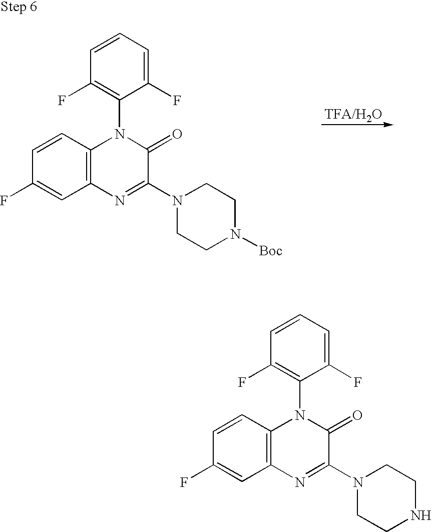

4-[4-(2,6-difluoro-phenyl)-7-fluoro-3-oxo-3,4-dihydro-quinoxalin-2-yl]-piperazine-1-carboxylic acid tert-butyl ester (0.515 g, 1.118 mmol) was combined with trifluoroacetic acid (2.24 ml) and water (0.22 ml). The reaction was stirred at RT for 1 hr. The reaction was diluted with MeOH (5 ml) and passed through a 10 g Varian Mega Bond Elut SCX column prewashed with 5% AcOH/MeOH. The column was eluted first with MeOH until washings were pH neutral, then with 1.0M NH₃/MeOH to obtain product. The filtrate was concentrated, and the product washed with EtOAc and concentrated to dryness (3×). The solids were dissolved in EtOAc, filter through a plug of Celite, and concentrated to dryness. The solids were washed with hexanes/ether, filtered and dried under vacuum at 65° C. to give 0.317 g (79%) of desired product.

MP=146-147° C.

Analysis for C₁₈H₁₅F₃N₄O.0.03 C₄H₈O₂, Calcd: C, 59.96; H, 4.23; N, 15.44; F, 15.70. Found: C, 59.93; H, 4.15; N, 15.29; F, 15.95.

¹H NMR (400 MHz, DMSO-D6) δ ppm 2.79 (m, 4 H) 3.86 (m, 4 H) 6.54 (dd, J=9.03, 5.12 Hz, 1 H) 6.97 (td, J=8.72, 2.81 Hz, 1 H) 7.27 (dd, J=9.76, 2.93 Hz, 1 H) 7.45 (m, 2 H) 7.74 (tt, J=8.57, 6.56 Hz, 1 H) MS (APCl+): 361 (M+1, 100%)

### EXAMPLE 11

### 1-(2-Chloro-phenyl)-3-[1,4]diazepan-1-yl-1H-quinoxalin-2-one Maleate

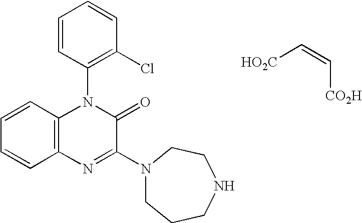

Following the procedures set forth in Example 1, but substituting (2-chloro-phenyl)-(2-nitro-phenyl)-amine for (4-fluoro-2-nitro-phenyl)-phenyl-amine in step 2, and substituting [1,4]diazepane-1-carboxylic acid tert-butyl ester for piperazine-1 -carboxylic acid tert-butyl ester in step 5, afforded the parent compound. The maleate salt was obtained by treating a 0.11 M solution of the parent compound in EtOAc with 1 equivalent of maleic acid in methanol (1 ml) in a 91% yield.

MP=159-161° C. Analysis for C₁₉H₁₉ClN₄O.C₄H₄O₄: C, 58.66; H, 4.92; N, 11.90; Cl, 7.53. Found: C, 58.63; H, 4.84; N, 11.85; Cl, 7.48.

### EXAMPLE 12

### 3-[1,4]Diazepan-1-yl-1-(3-fluoro-phenyl)-1H-quinoxalin-2-one

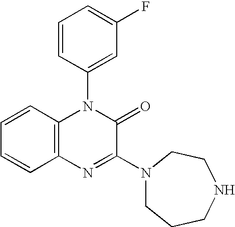

Following the procedures set forth in Example 1, but substituting (2-chloro-phenyl)-(2-nitro-phenyl)-amine for (4-fluoro-2-nitro-phenyl)-phenyl-amine in step 2, and substituting [1,4]diazepane-1-carboxylic acid tert-butyl ester for piperazine-1-carboxylic acid tert-butyl ester in step 5, afforded the title compound in similar yield.

MP=128-129° C. Analysis for C₁₉H₁₉FN₄O.0.06 C₄H₈O₂: C, 67.24; H, 5.71; N, 16.30; F, 5.53. Found: C, 67.18; H, 5.68; N, 16.17; F, 5.41.

### EXAMPLE 13

### 3-[1,4]Diazepan-1-yl-1-p-tolyl-1H-quinoxalin-2-one Maleate

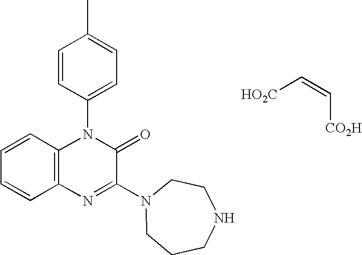

Following the procedures set forth in Example 1, but substituting (2-nitro-phenyl)-p-tolyl-amine for (4-fluoro-2-nitro-phenyl)-phenyl-amine in step 2, and substituting [1,4]diazepane-1-carboxylic acid tert-butyl ester for piperazine-1-carboxylic acid tert-butyl ester in step 5, afforded the parent compound. The maleate salt was obtained by treating a 0.1 OM solution of the parent compound in EtOAc with 1 equivalent of maleic acid in ethanol (1 ml) in a 91% yield.

MP=199-200° C. Analysis for C₂₀H₂₂N₄O.C₄H₄O₄: C, 63.99; H, 5.82; N, 12.44. Found: C, 63.83; H, 5.97; N, 12.21.

### EXAMPLE 14

### 3-[1,4]Diazepan-1-yl-6fluoro-1-phenyl-1H-quinoxalin-2-one

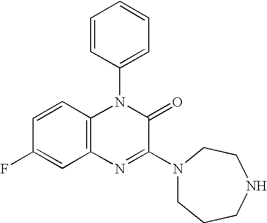

Following the procedures set forth in Example 1, but substituting [1,4]diazepane-1-carboxylic acid tert-butyl ester for piperazine-1-carboxylic acid tert-butyl ester in step 5, afforded the title compound in similar yield.

MP=151-152° C. Analysis for C₁₉H₁₉FN₄O.0.07 C₄H₈O₂.0.12 H₂0: C, 66.79; H, 5.76; N, 16.16; F, 5.48. Found: C, 66.42; H, 5.62; N, 16.02; F, 5.55.

### EXAMPLE 15

### 3-[1,4]Diazepan-1-yl-1-isopropyl-1H-quinoxalin-2-one Maleate

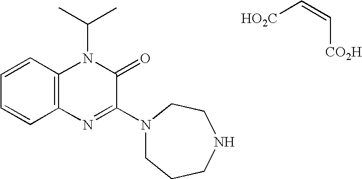

Following the procedures set forth in Example 1, but substituting isopropyl-(2-nitro-phenyl)-amine for (4-fluoro-2-nitro-phenyl)-phenyl-amine in step 2, and substituting [1,4]diazepane-1-carboxylic acid tert-butyl ester for piperazine-1-carboxylic acid tert-butyl ester in step 5, afforded the parent compound. The maleate salt was obtained by treating a 0.16M solution of the parent compound in EtOAc with 1 equivalent of maleic acid in methanol (1 ml) in a 85% yield.

MP=147-149° C. Analysis for C₁₆H₂₂N₄O.C₄H₄O₄: C, 59.69; H, 6.51; N, 13.92. Found: C, 59.46; H, 6.53; N, 13.87.

### EXAMPLE 16

### 1-(2,6-Difluoro-phenyl)-6-fluoro-1,4-dihydro-quinoxaline-2,3-dione

Step 1

Preparation of (2,6-Difluoro-phenyl)-(4-fluoro-2-nitro-phenyl)-amine

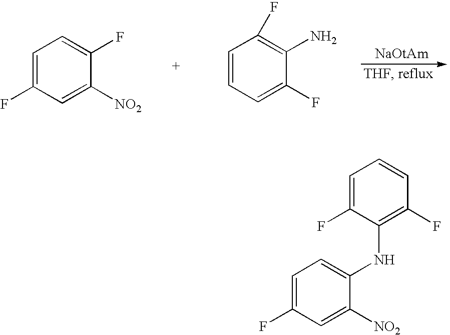

To a solution 496.5 mL (4.611 mol, 2.50 equivalents) of the 2,6-difluoroaniline in 1000 mL of dry THF under a nitrogen atmosphere was added 498 g (4.518 mol, 2.45 equivalents) of sodium tert-pentoxide in one portion. The temperature rose to 43° C., the reaction turned a dark purple color, and the temperature fell back to room temperature as the reaction was stirred for 2.5 hours. To the reaction was added 200 mL (1.844 mol, 1.00 equivalents) of the 2,5-difluoronitrobenzene dropwise. The reaction exothermed to reflux as the reagent was added and the addition rate was adjusted to maintain a calm reflux. The reaction was stirred for 1 hour after the addition was complete and confirmed to be complete by HPLC. The reaction was acidified with 1500 mL of 3M HCl and the phases separated. The organics were diluted with 2000 mL of IPA and atmospherically distilled until the distillate temperature reached 82° C as to remove all the THF. The product was precipitated by addition of 200 mL of water and cooling to −10° C. The precipitated solid was washed with 500 mL of IPA and dried to give 406.30 g (82.15%) of a dark brown solid that was 95.38% pure by HPLC.

¹H NMR (400 MHz, DMSO-d₆) d 6.64-6.69 (m, 1H), 7.21-7.29 (m, 2H), 7.36-7.48 (m, 2H), 7.92-7.96 (dd, J=3.13, 9.18, 1H), 9.03 (s, 1H)

19F NMR (376 MHz, DMSO-d₆) d 125.5 (m, 1F), −118.9 (m Hz, 2F)

Step 2

Preparation of (2,6-Difluoro-phenyl)-(4-fluoro-2-nitro-phenyl)-oxalamic Acid Ethyl Ester

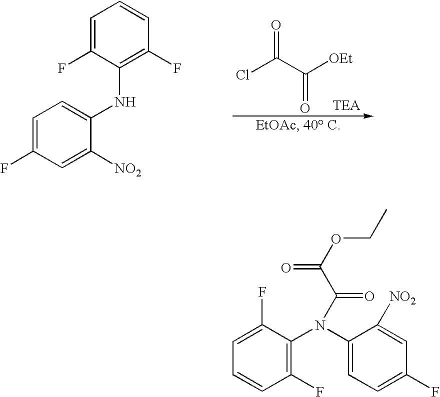

To a solution of 587.34 g (2.190 mol, 1.00 equivalents) of (2,6-Difluoro-phenyl)-4-fluoro-2-nitro-phenyl)amine in 2350 mL of EtOAc was added 915 mL (6.57 mol, 3.00 equivalents) of triethylamine. To the solution was added 367 mL (3.285 mol, 1.500 equivalents) of ethyl oxalyl chloride dropwise. The reaction exothermed so the addition rate was adjusted to maintain a temperature <40° C. The reaction was stirred for 1.5 hours after the addition and was confirmed to be complete by HPLC. The reaction was acidified to pH=1 with 3M HCl and the phases separated. The organic layer was taken on to the Raney nickel reduction-cyclization.

Step 3

Preparation of 1-(2,6-difluoro-phenyl)-6-fluoro-1,4-dihydro-quinoxaline-2,3-dione

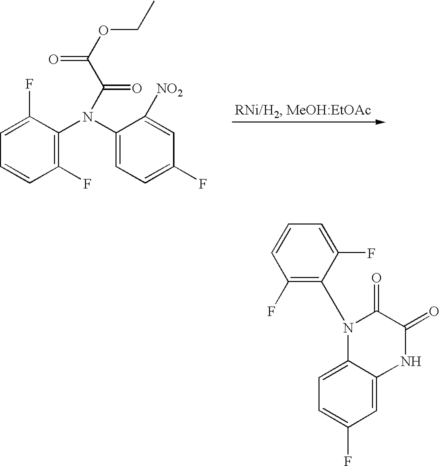

A solution of 205.00 g (556.67 mmol, 1.00 equivalents) of (2,6-Difluoro-phenyl)-(4-fluoro-2-nitro-phenyl)-oxalamic acid ethyl ester in 800 mL of EtOAc was diluted with an equal volume of MeOH then treated with 40 g 10% Pd catalyst, 55% water and the resulting slurry was shaken on a Parr hydrogenation apparatus at 50 PSI until hydrogen uptake had ceased (1.7 hrs). HPLC of a filtered sample indicated complete conversion of the starting material to the hydroxamic acid. The catalyst was removed by filtration and 100 g of sponge nickel catalyst was added. The resulting slurry was shaken in a Parr hydrogenation apparatus with heating at 50 C. at 50 PSI until HPLC indicated complete consumption of the intermediate hydroxamic acid and formation of the desired product. The reaction was vented, purged with nitrogen and carefully filtered. The solids were washed with additional warm MeOH and the filtrates were combined. The filtrates were concentrated to give a red solid which was slurried in 300 mL of DCM and filtered to give 135.10 g (83%) of an off-white solid.

¹H NMR (400 MHz, DMSO-d₆) d 6.64-6.68 (m,1 H), 7.21-7.28 (m, 2H), 7.37-7.48 (m, 2H), 7.92-7.95 (dd, J=3.03, 9.08, 1 H), 9.03 (s, 1 H)

19F NMR (376 MHz, DMSO-d6) d -125.5 (m,1 F), -118.9 (m Hz, 2F)

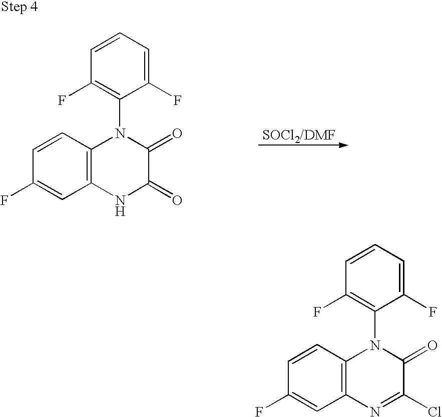

In a 3-L 3-neck flask equipped with a mechanical stirrer, thermocouple and addition funnel/condenser was slurried quinoxolone in CH2Cl2 (1500 mL). DMF (7.34 mL, 0.1 equiv) was added. Thionyl chloride (approx 10 mL) was added, resulting in the temperature increasing to 39° C., gas released. The remainder of the thionyl chloride (83 mL total) was added dropwise in three portions to maintain gas flow at a manageable rate, temperature at approximately 37° C. The reaction was stirred overnight, at which point it was still a slurry and was not complete (as judged by NMR analysis). The reaction was then heated to 40° C., at which point off-gassing resumed and refluxing and stirred for two hours. The reaction was still not complete (as judged by NMR analysis). Additional thionyl chloride (5 mL) was added and the reaction stirred another hour, at which point it was deemed complete. The thin green slurry was used directly in the next step.

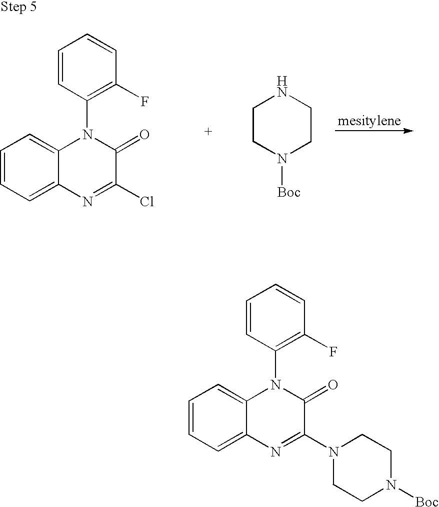

The piperazine was dissolved in CH2Cl2 (1000 mL). The iminoyl chloride solution from Step 4 was added dropwise over 30 minutes which resulted in a temperature increase from 15° C. to 38° C. The resulting thin slurry was stirred for two hours. HPLC analysis after 2 h showed reaction was complete. The reaction was cooled and held overnight. The reaction mixture was poured into an aqueous solution of potassium carbonate. The organic layer was extracted with 1 N HCl (2×1000 mL). The organic layer was distilled at atmospheric pressure (45° C.) until reaction volume reached approximately 2000 mL. Heptane (1500 mL) was added and the distillation temperature was gradually increased to 98° C., at which time the reaction volume was approximately 1500 mL. The reaction mixture was cooled slowly to room temperature and aged overnight. The resulting solids were isolated by filtration (420 g, 96% yield).

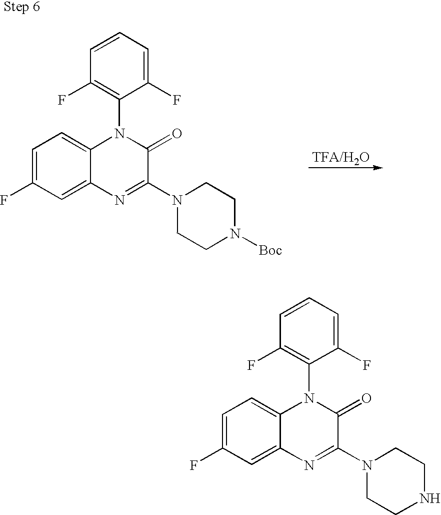

The reaction was performed reaction in a three necked jacketed reactor equipped with a thermocouple, addition funnel and mechanical stirrer. The jacket temperature was set to 25° C. The intermediate (400 g, 1.0 equiv) was dissolved in methylene chloride (2000 mL). Trifluoroacetic acid (268 mL, 4.0 equiv) was added dropwise in four portions, resulting in a temperature increase to approximately 29° C. The addition funnel was exchanged for a reflux condenser. The jacket temperature was increased to 43° C. (internal rose to 39° C.) and the reaction stirred overnight. The reaction was judged approximately 90% complete by HPLC and so was stirred an additional 24 hours, at which point the off-gassing had ceased and the reaction was judged complete by HPLC analysis. The reaction was cooled to 13° C. Potassium hydroxide (1000 mL of an approximately 25% w/w solution) was added dropwise in four portions, maintaining temperature below approximately 20° C. Methylene chloride (approximately 500 mL) and water (approximately 200 mL). The aqueous layer was removed. The organic layer was washed with water (approximately 1000 mL) and the aqueous layer was removed. The organic layer was distilled at atmospheric pressure until approximately 500 mL remained. Isopropanol (approximately 1500 mL) was added and the reaction was heated to 65° C. The distillation was maintained at this temperature by applying vacuum, with a final pressure of 250 torr. The distillation was continued until the solvent ratio was approximately 200:1 IPA:CH2Cl2 (total reaction volume was approximately 1200 mL). The reaction was diluted to 1.5 L total volume with IPA and then cooled to 50° C. @ 12° C./hour. Seeds were added (approximately 2 g) and the reaction was cooled to 0° C. @ 12° C./hour and stirred overnight. A sample was removed and PXRD analysis indicated form A. The reaction was then filtered and air was pulled through wet cake overnight. The wet cake was slurried in 9:1 water:IPA (1000 mL) and stirred for one hour at 25° C. The slurry was filtered and the resulting wet cake dried in the vacuum oven to afford PF-881003 (190 g, 61% yield) as an off white solid.

Pharmaceutical Composition Examples

In the following Examples, the term ‘active compound’ or ‘active ingredient’ refers to a compound according to the present invention above or in a suitable combination with another active agent for example an A2D ligand, an SRI an atypical antipsychotic, etc., and/or a pharmaceutically acceptable salt or solvate, according to the present invention.

(i) Tablet Compositions

The following compositions A and B can be prepared by wet granulation of ingredients (a) to (c) and (a) to (d) with a solution of povidone, followed by addition of the magnesium stearate and compression.

| | |
|---|---|
| mg/tablet | mg/tablet |

Composition A

| | | | |
|---|---|---|---|
| (a) | Active ingredient | 250 | 250 |
| (b) | Lactose B.P. | 210 | 26 |
| (c) | Sodium Starch Glycollate | 20 | 12 |
| (d) | Povidone B.P. | 15 | 9 |
| (e) | Magnesium Stearate | 5 | 3 |
| | | 500 | 300 |

Composition B

| | | | |
|---|---|---|---|
| (a) | Active ingredient | 250 | 250 |
| (b) | Lactose | 150 | 150 |
| (c) | Avicel PH 101 | 60 | 26 |
| (d) | Sodium Starch Glycollate | 20 | 12 |
| (e) | Povidone B.P. | 15 | 9 |
| (f) | Magnesium Stearate | 5 | 3 |
| | | 500 | 300 |

Composition C

| | mg/tablet |
|---|---|
| Active ingredient | 100 |
| Lactose | 200 |
| Starch | 50 |
| Povidone | 5 |
| Magnesium Stearate | 4 |
| | 359 |

The following compositions D and E can be prepared by direct compression of the admixed ingredients. The lactose used in formulation E is of the direct compression type.

mg/tablet

Composition D

| | |
|---|---|
| Active ingredient | 250 |
| Magnesium Stearate | 4 |
| Pregelatinised Starch NF15 | 146 |
| | 400 |

Composition E

| | |
|---|---|
| Active ingredient | 250 |
| Magnesium Stearate | 5 |
| Lactose | 145 |
| Avicel | 100 |
| | 500 |

Composition F (Controlled release composition)

| | |
|---|---|
| (a) Active ingredient | 500 |
| (b) Hydroxypropylmethylcellulose | 112 |
| (Methocel K4M Premium) | |
| (c) Lactose B.P. | 53 |
| (d) Povidone B.P.C. | 28 |
| (e) Magnesium Stearate | 7 |
| | 700 |

The composition can be prepared by wet granulation of ingredients (a) to (c) with a solution of povidone, followed by addition of the magnesium stearate and compression.

Composition G (Enteric-coated tablet)

Enteric-coated tablets of Composition C can be prepared by coating the tablets with 25 mg/tablet of an enteric polymer such as cellulose acetate phthalate, polyvinylacetate phthalate, hydroxypropylmethyl-cellulose phthalate, or anionic polymers of methacrylic acid and methacrylic acid methyl ester (Eudragit L). Except for Eudragit L, these polymers should also include 10% (by weight of the quantity of polymer used) of a plasticizer to prevent membrane cracking during application or on storage. Suitable plasticizers include diethyl phthalate, tributyl citrate and triacetin.

Composition H (Enteric-coated Controlled Release Tablet)

Enteric-coated tablets of Composition F can be prepared by coating the tablets with 50mg/tablet of an enteric polymer such as cellulose acetate phthalate, polyvinylacetate phthalate, hydroxypropylmethyl-cellulose phthalate, or anionic polymers of methacrylic acid and methacrylic acid methyl ester (Eudgragit L). Except for Eudgragit L, these polymers should also include 10% (by weight of the quantity of polymer used) of a plasticizer to prevent membrane cracking during application or on storage. Suitable plasticizers include diethyl phthalate, tributyl citrate and triacetin.

(ii) Capsule Compositions

Composition A

Capsules can be prepared by admixing the ingredients of Composition D above and filling two-part hard gelatin capsules with the resulting mixture. Composition B (infra) may be prepared in a similar manner.

mg/capsule

Composition B

| | | |
|---|---|---|
| (a) | Active ingredient | 250 |
| (b) | Lactose B.P. | 143 |
| (c) | Sodium Starch Glycollate | 25 |
| (d) | Magnesium Stearate | 2 |
| | | 420 |

Composition C

| | | |
|---|---|---|
| (a) | Active ingredient | 250 |
| (b) | Macrogol 4000 BP | 350 |
| | | 600 |

Capsules can be prepared by melting the Macrogol 4000 BP, dispersing the active ingredient in the melt and filling two-part hard gelatin capsules therewith.

| Composition D | mg/capsule |
|---|---|
| Active ingredient | 250 |
| Lecithin | 100 |
| Arachis Oil | 100 |
| | 450 |

Capsules can be prepared by dispersing the active ingredient in the lecithin and arachis oil and filling soft, elastic gelatin capsules with the dispersion.

Composition E (Controlled release capsule)

| | | mg/capsule |
|---|---|---|
| (a) | Active ingredient | 250 |
| (b) | Microcrystalline Cellulose | 125 |
| (c) | Lactose BP | 125 |
| (d) | Ethyl Cellulose | 13 |
| | | 513 |

The controlled release capsule formulation can be prepared by extruding mixed ingredients (a) to (c) using an extruder, then spheronising and drying the extrudate. The dried pellets are coated with a release controlling membrane (d) and filled into two-part, hard gelatin capsules.

| Composition F (Enteric capsule) | | | mg/capsule |
|---|---|---|---|
| | (a) | Active ingredient | 250 |
| | (b) | Microcrystalline Cellulose | 125 |
| | (c) | Lactose BP | 125 |
| | (d) | Cellulose Acetate Phthalate | 50 |
| | (e) | Diethyl Phthalat | 5 |
| | | | 555 |

The enteric capsule composition can be prepared by extruding mixed ingredients (a) to (c) using an extruder, then spheronising and drying the extrudate. The dried pellets are coated with an enteric membrane (d) containing a plasticizer (e) and filled into two-part, hard gelatin capsules.

Composition G (Enteric-coated Controlled Release Capsule)

Enteric capsules of Composition E can be prepared by coating the controlled-release pellets with 50 mg/capsule of an enteric polymer such as cellulose acetate phthalate, polyvinylacetate phthalate, hydroxypropylmethylcellulose phthalate, or anionic polymers of methacrylic acid and methacrylic acid methyl ester (Eudragit L). Except for Eudragit L, these polymers should also include 10% (by weight of the quantity of polymer used) or a plasticizer to prevent membrane cracking during application or on storage. Suitable plasticizers include diethyl phthalate, tributyl citrate and triacetin.

(iii) Intravenous injection composition

| | | |
|---|---|---|
| Active ingredient | 0.200 | g |
| Sterile, pyrogen-free phosphate buffer (pH 9.0) to | 10 | ml |

The active ingredient is dissolved in most of the phosphate buffer at 35-40° C., then made up to volume and filtered through a sterile micropore filter into sterile 10 ml glass vials (Type 1) which are sealed with sterile closures and overseals.

(iv) Intramuscular injection composition

| | | |
|---|---|---|
| Active ingredient | 0.20 | g |
| Benzyl Alcohol | 0.10 | g |
| Glycofurol 75 | 1.45 | g |
| Water for Injection q.s. to | 3.00 | ml |

The active ingredient is dissolved in the glycofurol. The benzyl alcohol is then added and dissolved, and water added to 3 ml. The mixture is then filtered through a sterile micropore filter and sealed in sterile 3 ml glass vials (Type 1).

(v) Syrup composition

| | | |
|---|---|---|
| Active ingredient | 0.25 | g |
| Sorbitol Solution | 1.50 | g |
| Glycerol | 1.00 | g |
| Sodium Benzoate | 0.005 | g |
| Flavour | 0.0125 | ml |
| Purified Water q.s. to | 5.0 | ml |

The sodium benzoate is dissolved in a portion of the purified water and the sorbitol solution added. The active ingredient is added and dissolved. The resulting solution is mixed with the glycerol and then made up to the required volume with the purified water.

| (vi) Suppository composition | mg/suppository |
|---|---|
| Active ingredient | 250 |
| Hard Fat, BP (Witepsol H15 - Dynamit NoBel) | 1770 |
| | 2020 |

One-fifth of the Witepsol H15 is melted in a steam-jacketed pan at 45° C. maximum. The active ingredient is sifted through a 200 lm sieve and added to the molten base with mixing, using a Silverson fitted with a cutting head, until a smooth dispersion is achieved. Maintaining the mixture at 45° C., the remaining Witepsol H15 is added to the suspension which is stirred to ensure a homogenous mix. The entire suspension is then passed through a 250 lm stainless steel screen and, with continuous stirring, allowed to cool to 40° C. At a temperature of 38-40° C., 2.02 g aliquots of the mixture are filled into suitable plastic moulds and the suppositories allowed to cool to room temperature.

| (vii) Pessary composition | mg/pessary |
|---|---|
| Active ingredient (63 lm) | 250 |
| Anhydrous Dextrose | 380 |
| Potato Starch | 363 |
| Magnesium Stearate | 7 |
| | 1000 |

The above ingredients are mixed directly and pessaries prepared by compression of the resulting mixture.

| (viii) Transdermal composition | | |
|---|---|---|
| Active ingredient | 200 | mg |
| Alcohol USP | 0.1 | ml |

Hydroxyethyl Cellulose

The active ingredient and alcohol USP are gelled with hydroxyethyl cellulose and packed in a transdermal device with a surface area of 10 cm².

All references cited herein are incorporated by reference in their entirety and for all purposes.

What is claimed is:

1. A compound of Formula I 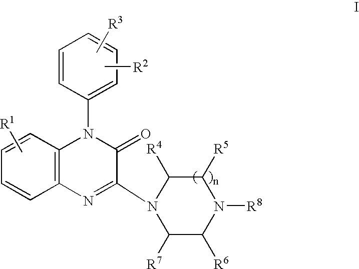
and pharmaceutically acceptable salts or derivatives thereof, wherein:
R¹ is H, (C₁-C₆)alkyl, or halogen;
R² and R³ are independently selected from is H, (C₁-C₆)alkyl, or halogen;
n is 1 or 2;
R⁴-R⁸ are independently selected from H and (C₁-C₈)alkyl.

2. A compound according to claim 1, wherein R² is a halogen.

3. A compound according to claim 1, wherein R² and R³ are fluoro.

4. A compound according to claim 1, wherein R¹ is fluoro, n is 1, and R⁸=H.

5. A compound according to claim 1, wherein R¹ is fluoro, R² and R³ are fluoro, n is 1, and R⁸=H.

6. A compound according to claim 1 and selected from:
1-Phenyl-3-piperazin-1-yl-1H-quinoxalin-2-one hydrochloride;
1-(2-Fluoro-phenyl)-3-piperazin-1-yl-1H-quinoxalin-2-one hydrochloride;
1-(2-Chloro-phenyl)-3-piperazin-1-yl-1H-quinoxalin-2-one;
3-Piperazin-1-yl-1-o-tolyl-1H-quinoxalin-2-one;
1-(3-Fluoro-phenyl)-3-piperazin-1-yl-1H-quinoxalin-2-one;
3-Piperazin-1-yl-1-p-tolyl-1H-quinoxalin-2-one maleate;
7-Fluoro-1-phenyl-3-piperazin-1-yl-1H-quinoxalin-2-one;
7-Methyl-1-phenyl-3-piperazin-1-yl-1H-quinoxalin-2-one;
6-Fluoro-1-phenyl-3-piperazin-1-yl-1H-quinoxalin-2-one;
1-(2,6-Difluoro-phenyl)-6-fluoro-3-piperazin-1-yl-1H-quinoxalin-2-one;
1-(2-Chloro-phenyl)-3-[1,4]diazepan-1-yl-1H-quinoxalin-2-one maleate;
3-[1,4]Diazepan-1-yl-1-(3-fluoro-phenyl)-1H-quinoxalin-2-one;
3-[1,4]Diazepan-1-yl-1-p-tolyl-1H-quinoxalin-2-one maleate;;
3-[1,4]Diazepan-1-yl-6-fluoro-1-phenyl-1H-quinoxalin-2-one; and
3-[1,4]Diazepan-1-yl-1-isopropyl-1H-quinoxalin-2-one maleate.

7. A pharmaceutical composition comprising a compound as defined in claim 1 and a pharmaceutically acceptable carrier.

8. A method of treating a disorder or condition selected from the group consisting of norepinephrine dysfunction, single episodic or recurrent major depressive disorders, dysthymic disorders, depressive neurosis and neurotic depression, melancholic depression including anorexia, weight loss, insomnia, early morning waking or psychomotor retardation; atypical depression or reactive depression including increased appetite, hypersomnia, psychomotor agitation or irritability, seasonal affective disorder and pediatric depression; bipolar disorders or manic depression, for example, bipolar I disorder, bipolar II disorder and cyclothymic disorder; conduct disorder; attention deficit hyperactivity disorder (ADHD); disruptive behavior disorder; behavioral disturbances associated with mental retardation, autistic disorder, and conduct disorder; anxiety disorders such as panic disorder with or without agoraphobia, agoraphobia without history of panic disorder, specific phobias including specific animal phobias, social anxiety, social phobia including social anxiety disorder, obsessive-compulsive disorder and related spectrum disorders, stress disorders including post-traumatic stress disorder, acute stress disorder and chronic stress disorder, and generalized anxiety disorders; borderline personality disorder; schizophrenia and other psychotic disorders including schizophreniform disorders, schizoaffective disorders, delusional disorders, brief psychotic disorders, shared psychotic disorders, psychotic disorders with delusions or hallucinations, psychotic episodes of anxiety, anxiety associated with psychosis, psychotic mood disorders such as severe major depressive disorder; mood disorders associated with psychotic disorders such as acute mania and depression associated with bipolar disorder; mood disorders associated with schizophrenia; delirium, dementia, and amnestic and other cognitive or neurodegenerative disorders, such as Parkinson's disease (PD), Huntington's disease (HD), Alzheimer's disease, senile dementia, dementia of the Alzheimer's type, memory disorders, loss of executive function, vascular dementia, and other dementias due to HIV disease, head trauma, Parkinson's disease, Huntington's disease, Pick's disease, Creutzfeldt-Jakob disease, or due to multiple etiologies; movement disorders such as akinesias, dyskinesias, including familial paroxysmal dyskinesias, spasticities, Tourette's syndrome, Scott syndrome, PALSYS and akinetic-rigid syndrome; extra-pyramidal movement disorders such as medication-induced movement disorders, for example, neuroleptic-induced Parkinsonism, neuroleptic malignant syndrome, neuroleptic-induced acute dystonia, neuroleptic-induced acute akathisia, neuroleptic-induced tardive dyskinesia and medication-induced postural tremour; addictive disorders and withdrawal syndrome, chemical dependencies and addictions including dependencies on, or addictions to, alcohol, heroin, cocaine, benzodiazepines, sychoactive substances, nicotine, or phenobarbitol and behavioral addictions including addiction to gambling; ocular disorders such as glaucoma and ischemic retinopathy addictive disorders including those due to alcohol, nicotine, and other psychoactive substances and withdrawal syndrome, adjustment disorders including depressed mood, anxiety, mixed anxiety and depressed mood, disturbance of conduct, and mixed disturbance of conduct and mood; age-associated learning and mental disorders including Alzheimer's disease; anorexia nervosa; apathy; attention-deficit or other cognitive disorders due to general medical conditions including attention-deficit disorder (ADD) and attention-deficit hyperactivity disorder (ADHD) and it's recognized sub-types; bulimia nervosa; chronic fatigue syndrome; pain; chronic pain; cyclothymic disorder; depression including adolescent depression and minor depression; fibromyalgia and other somatoform disorders including somatization disorder; conversion disorder; pain disorder; hypochondriasis; body dysmorphic disorder; undifferentiated somatoform disorder; and somatoform NOS; incontinence including stress incontinence; genuine stress incontinence; and mixed incontinence; urinary disorders; premature ejaculation; inhalation disorders; intoxication disorders including alcohol addiction; mania; migraine headaches; obesity including reducing the weight of obese or overweight patients; restless legs syndrome; oppositional defiant disorder; peripheral neuropathy; diabetic neuropathy; post-herpetic neuralgic; premenstrual dysphoric disorder including premenstrual syndrome and late luteal phase dysphoric disorder; hot flashes; sleep disorders including narcolepsy, insomnia and enuresis; specific developmental disorders; selective serotonin reuptake inhibition (SSRI) “poop out” syndrome; and TIC disorders including Tourette's Disease in a mammal, including a human, comprising administering to a mammal in need of such treatment an amount of a compound of the formula I as defined in claim 1.

9. A method of treatment of attention deficit hyperactivity disorder, urinary disorders, pain, premature ejaculation, or fibromyalgia, which comprises administering a therapeutically effective amount of a compound of claim 1.

10. A compound selected from:
(2,6-Difluoro-phenyl)-(4-fluoro-2-nitro-phenyl)-amine;
(2,6-Difluoro-phenyl)-(4-fluoro-2-nitro-phenyl)-oxalamic acid ethyl ester; and
1-(2,6-difluoro-phenyl)-6-fluoro-1,4-dihydro-quinoxaline-2,3-dione.
